(19) United States
(12) Patent Application Publication
(10) Pub. No.: US 2025/0114456 A1
(43) Pub. Date: Apr. 10, 2025

(54) ENHANCED IMMUNOTHERAPEUTIC METHOD COMPRISING COMBINED ADMINISTRATION OF PHOTODYNAMIC / RADIOTHERAPEUTIC COMPOUNDS AND IMMUNOTHERAPEUTIC AGENTS

(71) Applicant: Theralase Technologies, Inc., Toronto (CA)

(72) Inventors: Arkady MANDEL, North York (CA); Roger DUMOULIN-WHITE, Toronto (CA)

(21) Appl. No.: 18/480,739

(22) Filed: Oct. 4, 2023

(51) Int. Cl. A61K 41/00; A61K 39/00; A61K 39/395; A61P 35/00

(52) U.S. Cl. CPC A61K 41/0057; A61K 39/0011; A61K 39/3955; A61P 35/00

(57) ABSTRACT

Disclosed is a method for treating a condition associated with hyperproliferating cells, the method including the steps of (a) administering to a patient having the condition an immunotherapeutic agent in an anti-tumor effective amount; (b) administering to the patient a photodynamic compound; and (c) administering light and/or ionizing radiation to the patient in whom the immunotherapeutic agent and the photodynamic compound have been administered to treat the condition.

### BACKGROUND OF THE INVENTION

### 1. Field of Invention

This invention relates to photodynamic, radiotherapeutic and immunotherapeutic methods for treating cancer.

### 2. Description of Related Art

Photodynamic therapy (“PDT”) is currently an active area of research for the treatment of diseases associated with hyperproliferating cells such as cancer and non-malignant lesions. The development of new photodynamic compounds (“PDCs”) for PDT has been increasingly focused on metallo-supramolecular complexes derived from metals. For example, WO 2013158550 A1 and WO 2014145428 A2 disclose metal based PDCs useful as in vivo diagnostic agents, as therapeutic agents for treating or preventing diseases that involve unwanted and/or hyperproliferating cell etiology (including cancer) as agents for treating infectious diseases and as agents for pathogen disinfection and/or sterilization. U.S. Pat. Nos. 6,962,910, 7,612,057, 8,445,475 and 8,148,360 disclose supramolecular metal complexes capable of cleaving DNA when irradiated by low energy visible light with or without molecular oxygen.

Transferrin associated TLD-1433 (i.e., RUTHERRIN, Theralase Technologies Inc., Toronto, Canada) administered to cancer patients is preferentially absorbed via the Transferrin Receptor (“TfR”) into bladder cancer cells, leaving healthy urothelial cells intact. See, e.g., Lilge et al. “Evaluation of a Ruthenium coordination complex as photosensitizer for PDT of bladder cancer: Cellular response, tissue selectivity and in vivo response.” Translational Biophotonics 2, no. 1-2 (2020): e201900032. The absorbed PDC is activated by light and/or Radiation Therapy (“RT”), such as X-rays to produce singlet oxygen or Reactive Oxygen Species (“ROS”)) to induce oxidative stress and hence apoptosis of the cancer cell. This PDT acts as the primary Mechanism Of Action (“MOA”) of destruction of cancer cells. A secondary action of PDT is that when the cancer cells dies by apoptosis, it releases its cellular contents into the blood stream through Immunogenic Cell Death (“ICD”); thereby, informing the immune system of its demise and activating the immune system to destroy any remaining cancer cells of similar biological structure.

Immunotherapy is an alternative method for treating cancer, in which the immune system of a cancer patient is primarily activated to destroy cancer cells. Immunotherapy refers to treatments that stimulate, enhance or suppress the body's own immune system, such as: CAR T-cell therapies, oncolytic viruses, immune checkpoint inhibitors and monoclonal antibodies, to name a few. Immunotherapy works by targeting proteins or receptors on cancer cells (called antigens) to unveil them to the immune system, so the immune system can destroy them or in the case of oncolytic viruses to allow the proliferation of the virus to destroy the cancer cells in question.

For example, Chimeric Antigen Receptor (“CAR”) T-cell therapy boosts the effects of specialized white blood cells, known as T-cells. The US. Food and Drug Administration (“FDA”) has approved six CAR T-cell therapies, which are listed in Table 1 below along with patents disclosing same.

TABLE 1
Patent Publications relating to certain U.S. Food and Drug
Administration (FDA) approved CAR T-cell therapies.

| Generic Name | Brand Name | Related Publication |
|---|---|---|
| Idecabtagene Vicleucel | ABECMA | U.S. Pat. No. 9,765,342 |
| Lisocabtagene Maraleucel | BREYANZI | U.S. Pat. No. 7,446,190 |
| Tisagenlecleucel | KYMRIAH | U.S. Pat. No. 8,911,993 |
| Brexucabtagene Autoleucel | TECARTUS | U.S. Pat. No. 9,855,298; |
| | | U.S. Pat. No. 10,322,146 |
| Axicabtagene Ciloleucel | YESCARTA | U.S. Pat. No. 9,855,298; |
| | | U.S. Pat. No. 10,322,146 |
| Ciltacabtagene Autoleucel | CARVYKTI | US 20220265710 A1 |

Cancer vaccines work by boosting the immune system to identify and destroy antigens on cancer cells. For example, cancer vaccines can promote cytotoxic CD8 positive T-cells.

Oncolytic virus therapy infects cancer cells with virus particles and/or non-replicated viral vectors to destroy the cells.

Immune Checkpoint Inhibitors (“ICIs”) are immunotherapeutic agents that inhibit the ability of immune checkpoints to downregulate immune responses, thus enabling the immune system to respond more strongly to cancer.

Monoclonal antibodies (“MABs”) are immune system proteins designed to bind to specific targets. Certain MABs function as ICIs to downregulate or block checkpoint proteins on the surface of immune cells (T and B cells) or downregulate or block checkpoint proteins on the surface of cancer cells, so that the immune system can identify, attack and kill cancer cells. These checkpoint proteins are located either on the surface of the immune cells (Programmed Cell Death Protein 1 (“PD-1”)) or on cancer cells (Programmed Death Ligand 1 (“PD-L1”) or Programmed Death Ligand 2 (“PD-L2”)).

U.S. Pat. No. 10,155,037 B2 discloses anti-PD-1 antibodies, which are useful alone or in combination with other therapeutics to treat cancer and other diseases.

U.S. Pat. No. 10,618,916 B2 discloses compounds which modulate PD-1/PD-L1 protein/protein interaction and are useful in the treatment of various diseases including infectious diseases and cancer.

Examples of MABs useful for immunotherapy are listed in the following table along with patents which relate to preparing and/or using said drugs and other immunotherapy drugs.

TABLE 2

| Generic Name | Brand Name | Related Publication |
|---|---|---|
| Atezolizumab | TECENTRIQ | U.S. Pat. No. 8,217,149 |
| Avelumab | BAVENCIO | U.S. Pat. No. 9,624,298 |
| Dostarlizumab | JEMPERLI | U.S. Pat. No. 9,815,897 |
| Durvalumab | IMFINZI | U.S. Pat. No. 9,493,565 |
| Ipilimumab | YERVOY | U.S. Pat. No. 6,984,720 |
| Nivolumab | OPDIVO | U.S. Pat. No. 8,008,449 |
| Pembrolizumab | KEYTRUDA | U.S. Pat. No. 8,354,509 |
| Cetrelimab | N/A | US20190225689A1 |
| Sasanlimab | N/A | U.S. Pat. No. 10,155,037B2 |
| Pucotenlimab | PUYOUHENG | US20190040138A1 |
| Bevacizumab | AVASTIN | U.S. Pat. No. 6,407,213B1 |
| Trastuzumab | HERCEPTIN | U.S. Pat. No. 6,627,196B1 |
| Onartuzumab | N/A | U.S. Pat. No. 7,572,897B2 |
| Erdafitinib | BALVERSA | U.S. Pat. No. 8,895,601B2 |
| Enapotamab | ENAV | US20180214549A1 |
| Daratumumab | DARZALEX | U.S. Pat. No. 7,829,673B2 |

To date, such immunotherapy drugs have delivered stellar results for some cancer patients and zero benefit for others, with only 20 to 40% of patients deriving benefit, based primarily on the type of antigen that presents on an individual patient's cancer cells. If the patient's cancer cells do not have a specific antigen, the immunotherapy drugs prescribed will have no benefit. They can also be associated with serious or life-threatening side-effects.

ICIs are limited in effectiveness for patients who lack the specific antigen (protein) on their cancer cells). The immune response thus remains incompletely activated or is suppressed in many types of tumors, limiting the effectiveness of ICIs. In patients harboring these types of tumors, increased antitumor activity cannot be established from PD-1, PD-L1 or PD-L2 inhibition alone. See Bardhan et al. “The PD1: PD-L1/2 pathway from discovery to clinical implementation.” Frontiers in immunology 7 (2016): 550.

It is therefore desired to provide additional compounds, compositions and therapeutic methods for treating cancer that do not possess these limitations in expressed antigens.

It is further desired to provide additional compounds, compositions and therapeutic methods for treating cancer which combine the benefits of immunotherapy and photodynamic/radiotherapeutic therapy.

All references cited herein are incorporated herein by reference in their entireties.

### BRIEF SUMMARY OF THE INVENTION

Accordingly, a first aspect of the invention is a method for treating a condition associated with hyperproliferating cells, said method comprising the steps of:
(a) administering to a patient having the condition an immunotherapeutic agent in an anti-tumor effective amount;
(b) administering to the patient a photodynamic compound; and
(c) administering radiation to the patient in whom the immunotherapeutic agent and the photodynamic compound have been administered to treat the condition.

In certain embodiments, the condition is cancer.

In certain embodiments, the immunotherapeutic agent is an anti-PD-1 monoclonal antibody, an anti-PD-L1 monoclonal antibody or an anti-PD-L2 monoclonal antibody.

In certain embodiments, the immunotherapeutic agent is a cancer vaccine.

In certain embodiments, the immunotherapeutic agent is at least one member selected from the group consisting of: idecabtagene vicleucel, lisocabtagene maraleucel, tisagenlecleucel, brexucabtagene autoleucel, axicabtagene ciloleucel, ciltacabtagene autoleucel, atezolizumab, avelumab, dostarlizumab, durvalumab, ipilimumab, nivolumab, pembrolizumab, pucotenlimab, pidilizumab, cemiplimab, spartalizumab, camrelizumab, cetrelimab, toripalimab, sintilimab, sasanlimab, dostarlizumab, SHR-1210, PDR001, MGA012, PDR001, AB122, AMP-224, JTX-4014, BGB-108, BCD-100, BAT1306, LZM009, AK105, HLX10, bevacizumab, trastuzumab, onartuzumab, erdafitinib, enapotamab, daratumumab, FAZ053, KN035, CS1001, SHR-1316, CBT-502, A167, STI-A101, CK-301, BGB-A333, MSB-2311, HLX20, LY3300054, sipuleucel-T, Bacillus Calmette-Guérin vaccine, nadofaragene firadonevec and T-VEC.

In certain embodiments, the photodynamic compound is represented by one of the following formulas:

(i) formula (I):

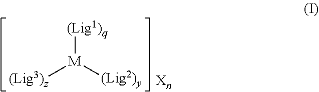

including hydrates, solvates, pharmaceutically acceptable salts, prodrugs and complexes thereof, wherein:
M at each occurrence is independently selected from the group consisting of osmium, ruthenium and rhodium;
X is selected from the group consisting of Cl⁻, PF₆⁻, Br⁻, BF₄⁻, ClO₄⁻, CF₃SO₃⁻, and SO₄⁻²;
n=0, 1, 2, 3, 4, or 5;
q is independently at each occurrence 0, 1, or 2;
y is independently at each occurrence 0, 1, or 2;
z is independently at each occurrence 1, 2, or 3;
Lig¹ is a bidentate ligand that at each occurrence is each independently selected from the group consisting of

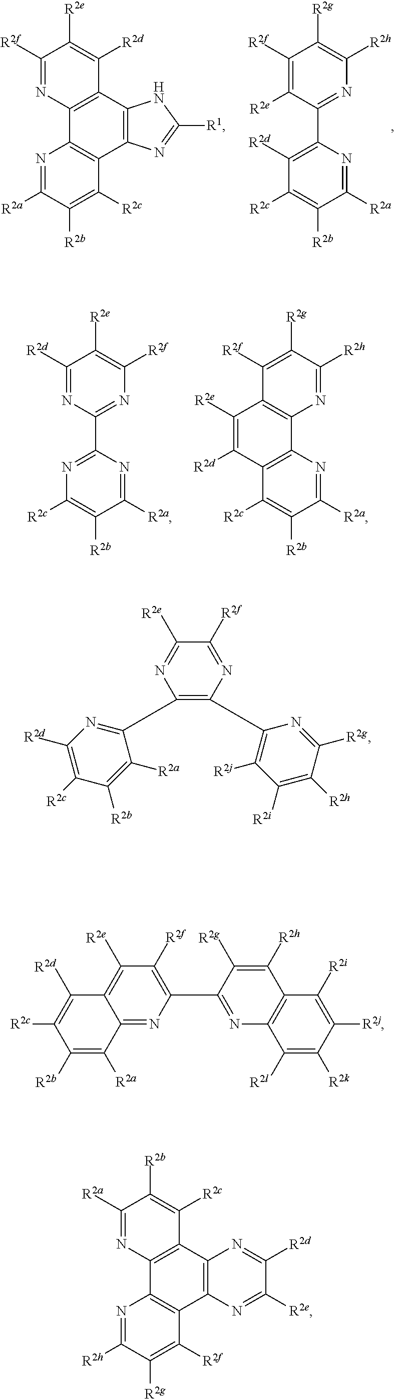

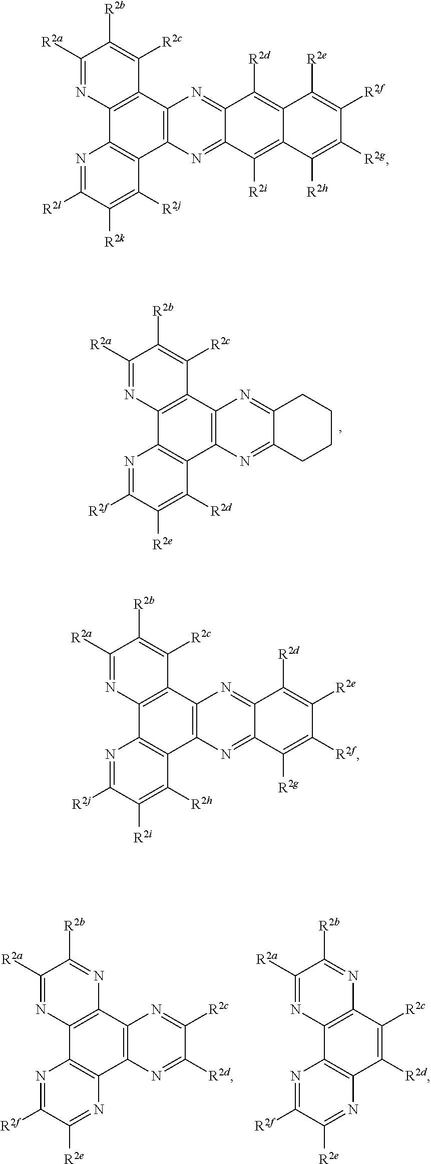

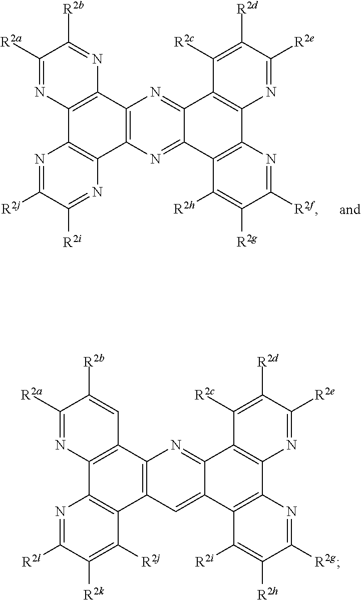

Lig² is a bidentate ligand that at each occurrence is each independently selected from the group consisting of

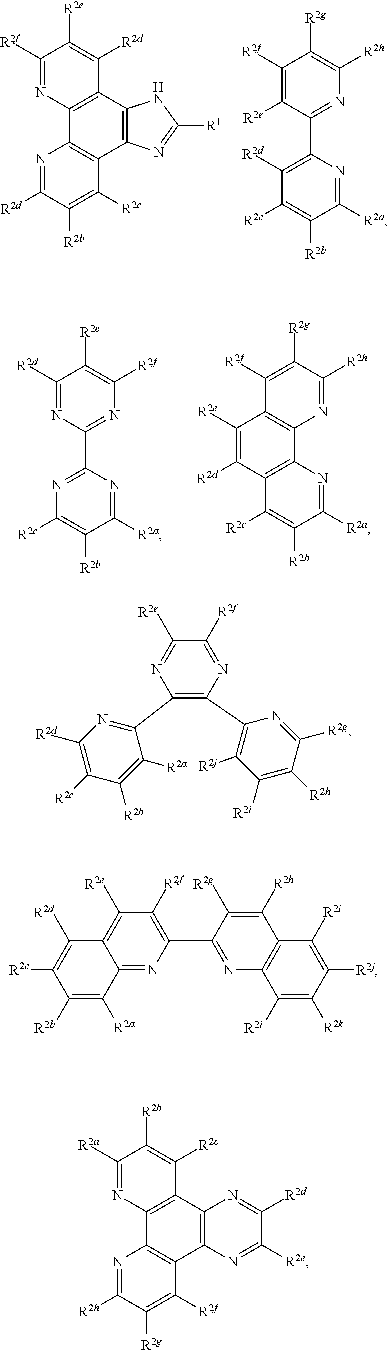

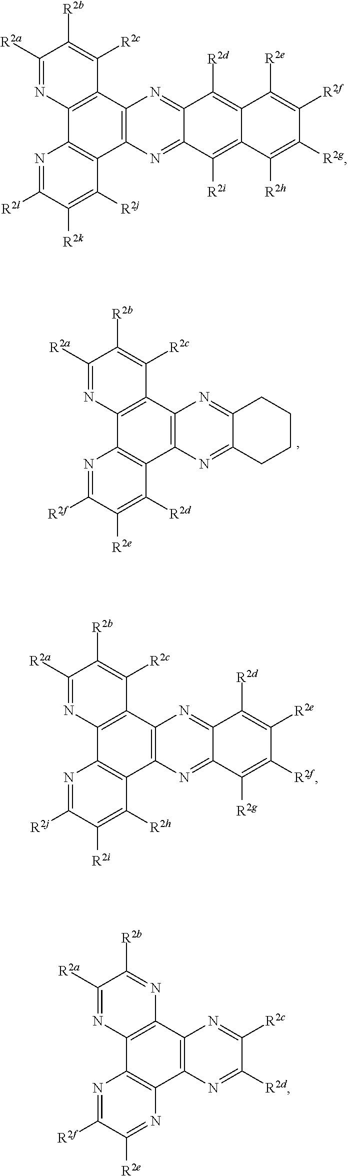

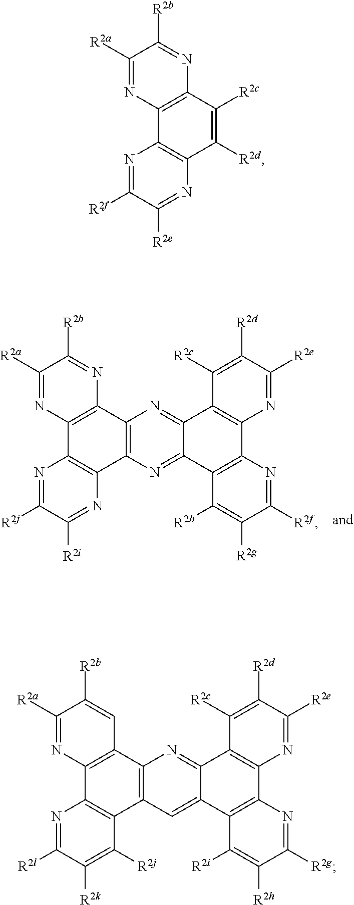

Lig³ is a bidentate ligand that at each occurrence is each independently selected from the group consisting of

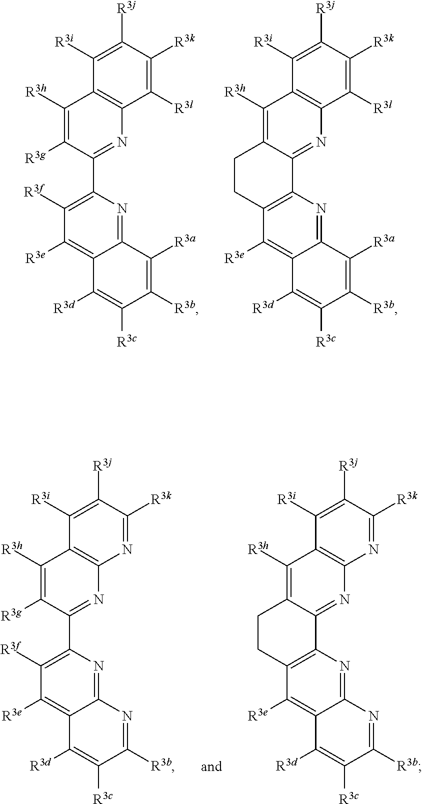

R¹ is selected from the group consisting of hydrogen, optionally substituted phenyl, optionally substituted aryl, optionally substituted heteroaryl, 4-pyridyl, 3-pyridyl, 2-thiazole, 2-pyrolyl, 2-furanyl,

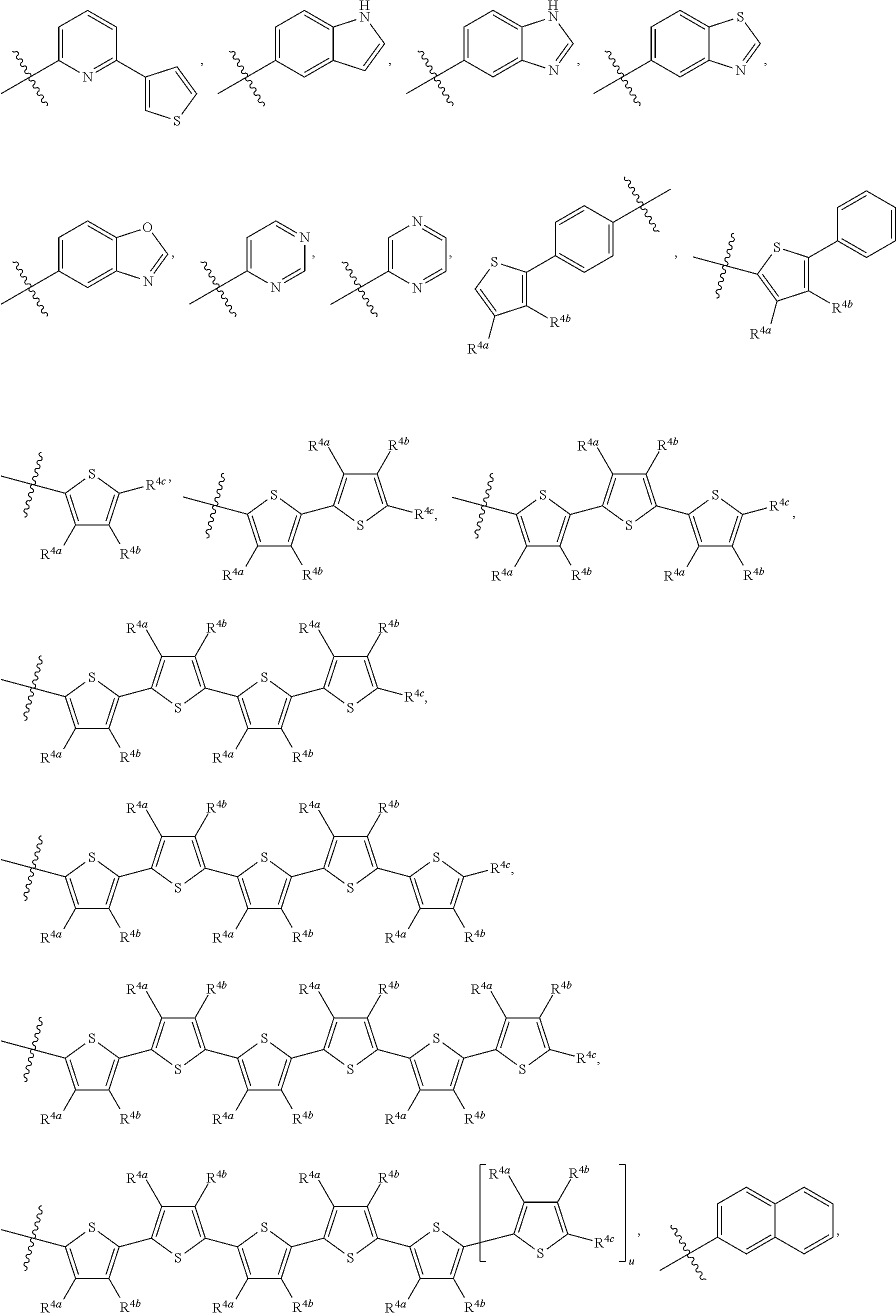

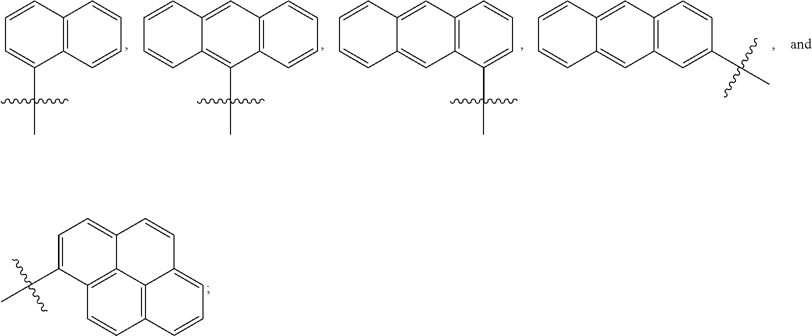

u is an integer;
R^{2a}, R^{2b}, R^{2c}, R^{2d}, R^{2e}, R^{2f}, R^{2g}, R^{2h}, R²ⁱ, R^{2j}, R^{2k}, and R^{2l} at each occurrence are each independently selected from the group consisting of hydrogen, C₁₋₆ optionally substituted alkyl, C₁₋₆ optionally substituted branched alkyl, C₃₋₇ optionally substituted cycloalkyl, C₁₋₆ optionally substituted haloalkyl, C₁₋₆ optionally substituted alkoxy, CO₂R⁵, CONR⁶₂, NR⁷₂, SO₃H, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h} R³ⁱ, R^{3j}, R^{3k}, and R^{3l} at each occurrence are each independently selected from the group consisting of hydrogen, C₁₋₆ optionally substituted alkyl, C₁₋₆ optionally substituted branched alkyl, C₁₋₆ optionally substituted haloalkyl, C₁₋₆ optionally substituted alkoxy, optionally substituted phenyl, and CO₂R⁸;
R^{4a}, R^{4b}, and R^{4c} at each occurrence are each independently selected from the group consisting of hydrogen, C₁₋₆ optionally substituted alkyl, C₁₋₆ optionally substituted branched alkyl, C₁₋₆ optionally substituted cycloalkyl, C₁₋₆ optionally substituted haloalkyl, C₁₋₆ optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence are each independently selected from the group consisting of hydrogen and optionally substituted alkyl;
R⁶ at each occurrence are each independently selected from the group consisting of hydrogen and optionally substituted alkyl;
R⁷ at each occurrence are each independently selected from the group consisting of hydrogen and optionally substituted alkyl; and
R⁸ at each occurrence are each independently selected from the group consisting of hydrogen and optionally substituted alkyl.
(ii) formula (VI):

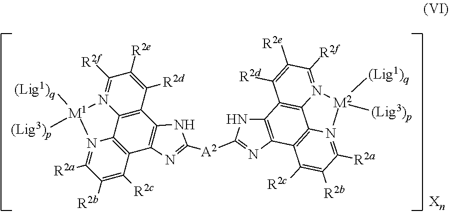

including hydrates, solvates, pharmaceutically acceptable salts, prodrugs and complexes thereof wherein;
M¹ and M² at each occurrence is independently selected from the group consisting of osmium, manganese, molybdenum, rhenium, ruthenium, iron, cobalt, rhodium, iridium, nickel, platinum, and copper;
A² is selected from the group consisting of

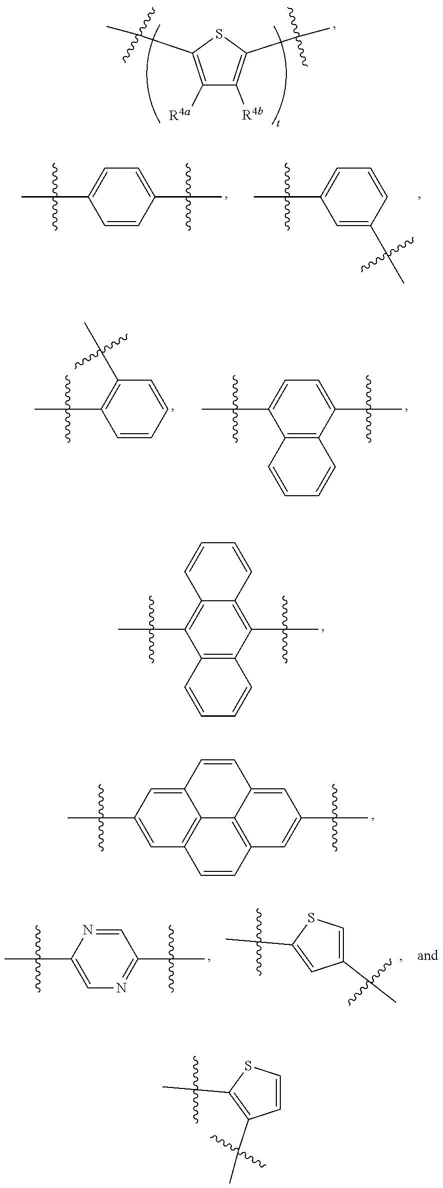

t is an integer;
(iii) formula (VIIa):

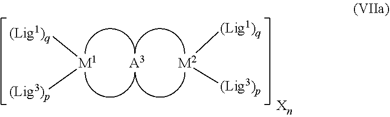

including hydrates, solvates, pharmaceutically acceptable salts, prodrugs and complexes thereof wherein:
A³ is selected from the group consisting of

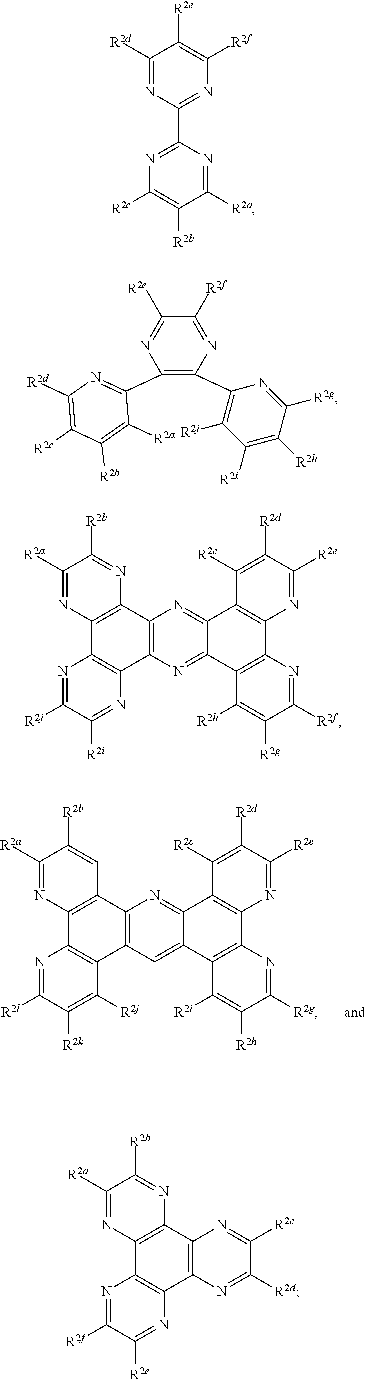

Lig¹ is a bidentate ligand that at each occurrence is each independently selected from the group consisting of

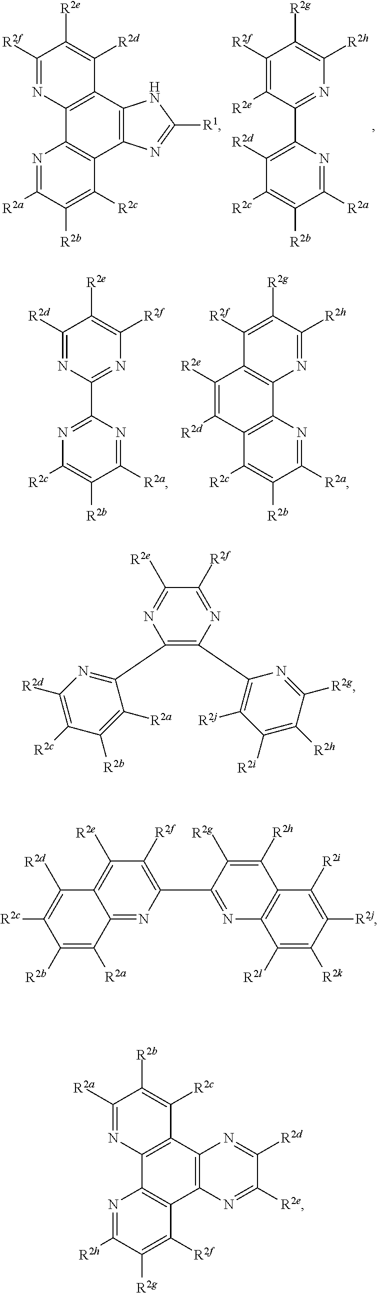

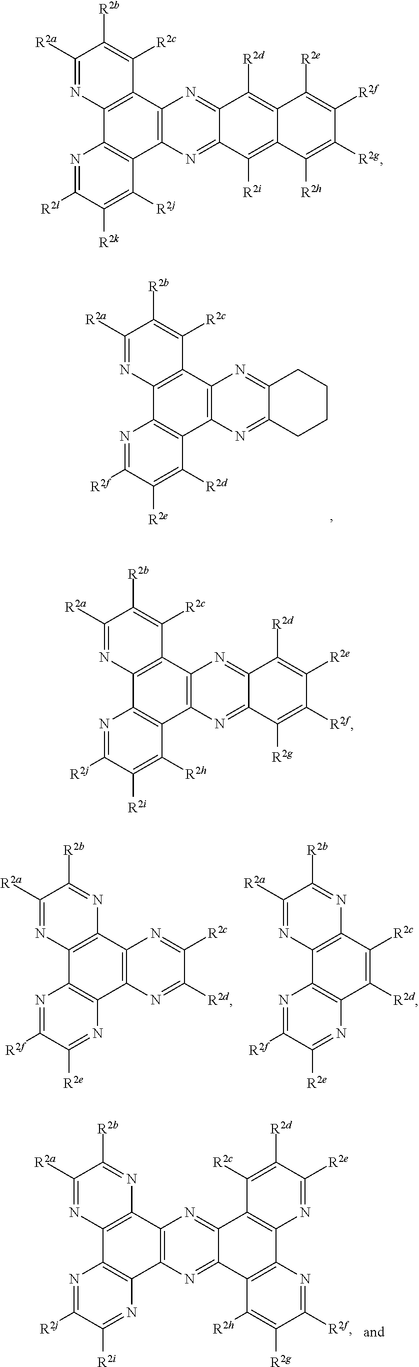

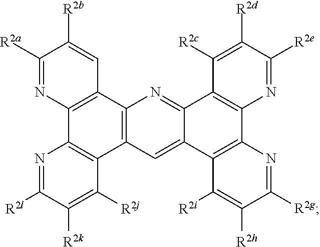

Lig³ is a bidentate ligand that at each occurrence is each independently selected from the group consisting of

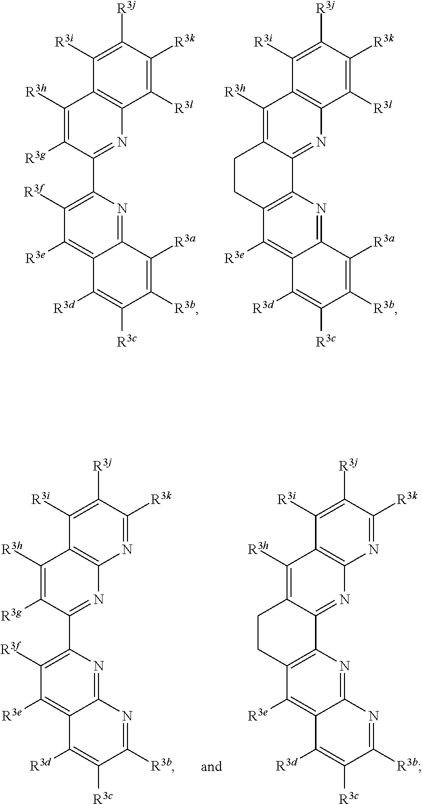

R¹ is selected from the group consisting of hydrogen, optionally substituted phenyl, optionally substituted aryl, optionally substituted heteroaryl, 4-pyridyl, 3-pyridyl, 2-thiazole, 2-pyrolyl, 2-furanyl,

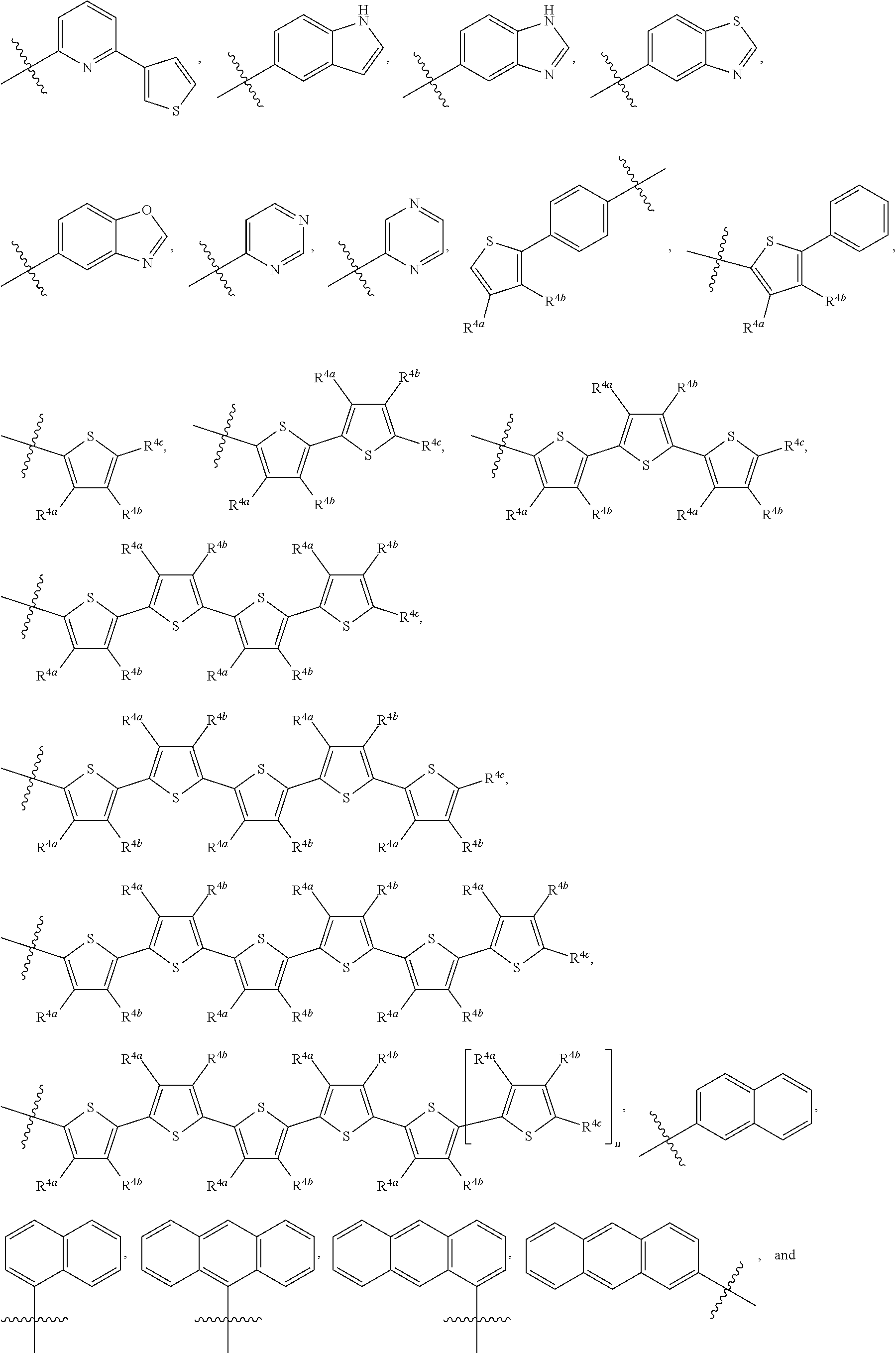

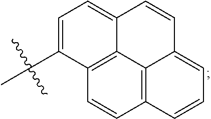 [SMILES: *c1ccc2ccc3cccc4ccc1c2c34]

u is an integer;
R^{2a}, R^{2b}, R^{2c}, R^{2d}, R^{2e}, R^{2f}, R^{2g}, R^{2h}, R²ⁱ, R^{2j}, R^{2k}, and R^{2l} at each occurrence are each independently selected from the group consisting of hydrogen, C₁₋₆ optionally substituted alkyl, C₁₋₆ optionally substituted branched alkyl, C₃₋₇ optionally substituted cycloalkyl, C₁₋₆ optionally substituted haloalkyl, C₁₋₆ optionally substituted alkoxy, CO₂R⁵, CONR⁶₂, NR⁷₂, SO₃H, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h} R³ⁱ, R^{3j}, R^{3k}, and R^{3l} at each occurrence are each independently selected from the group consisting of hydrogen, C₁₋₆ optionally substituted alkyl, C₁₋₆ optionally substituted branched alkyl, C₁₋₆ optionally substituted haloalkyl, C₁₋₆ optionally substituted alkoxy, optionally substituted phenyl, and CO₂R⁸;
R^{4a}, R^{4b}, and R^{4c} at each occurrence are each independently selected from the group consisting of hydrogen, C₁₋₆ optionally substituted alkyl, C₁₋₆ optionally substituted branched alkyl, C₁₋₆ optionally substituted cycloalkyl, C₁₋₆ optionally substituted haloalkyl, C₁₋₆ optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence are each independently selected from the group consisting of hydrogen and optionally substituted alkyl;
R⁶ at each occurrence are each independently selected from the group consisting of hydrogen and optionally substituted alkyl;
R⁷ at each occurrence are each independently selected from the group consisting of hydrogen and optionally substituted alkyl; and
R⁸ at each occurrence are each independently selected from the group consisting of hydrogen and optionally substituted alkyl
p is independently at each occurrence 0, 1, or 2;
q is independently at each occurrence 0, 1, or 2; and
n is 0, 1, 2, 3, 4, or 5.
(iv) formula (II):

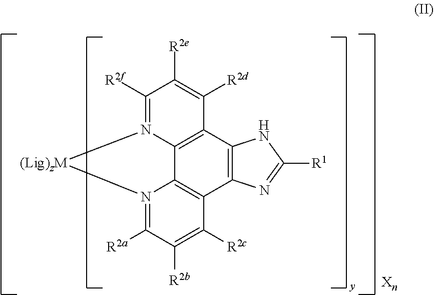

including hydrates, solvates, pharmaceutically acceptable salts, prodrugs and complexes thereof, wherein:
M is selected from the group consisting of manganese, molybdenum, rhenium, iron, ruthenium, osmium, cobalt, rhodium, iridium, nickel, platinum, and copper;
X is selected from the group consisting of Cl⁻, PF₆⁻, Br⁻, BF₄⁻, ClO₄⁻, CF₃SO₃⁻, and SO₄⁻²;
n=0, 1, 2, 3, 4, or 5;
y=1, 2, or 3;
z=0, 1, or 2;
Lig at each occurrence is independently selected from the group consisting of

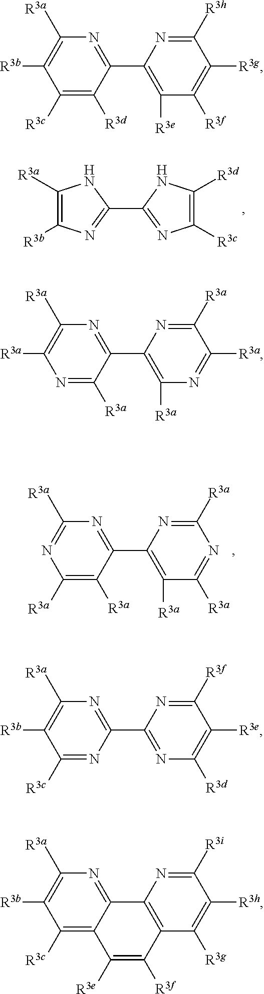

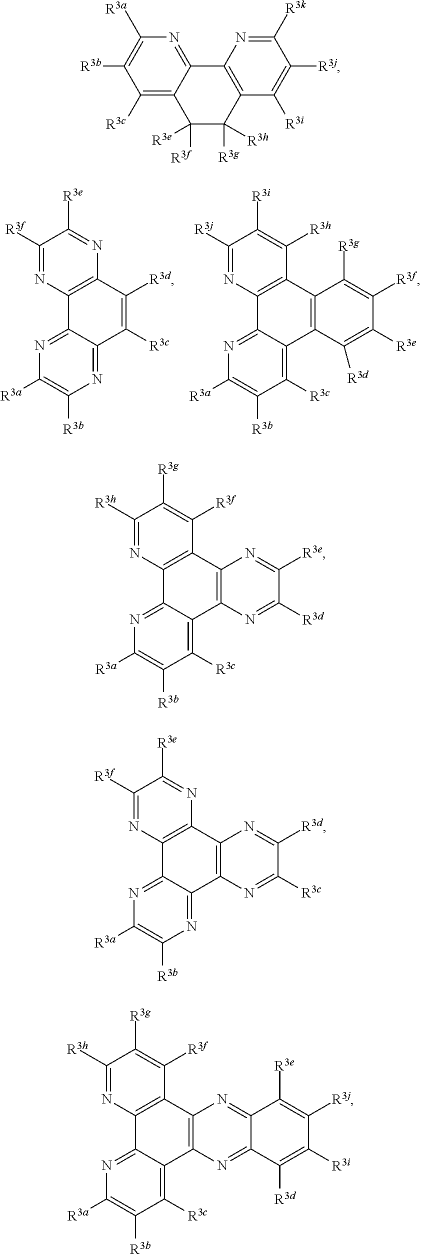

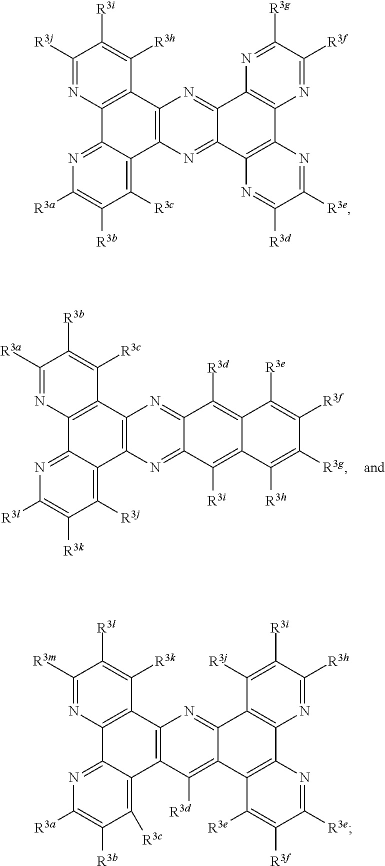

R¹ is selected from the group consisting of

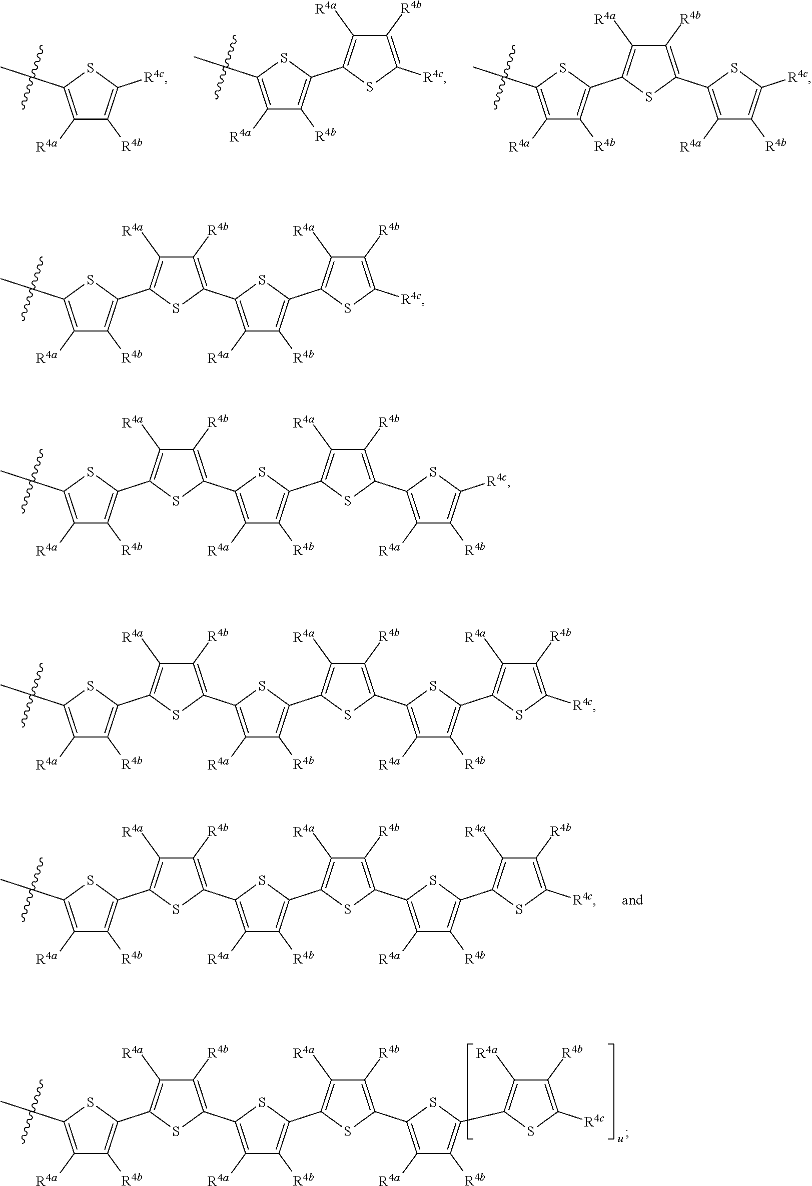

u is an integer;
R^{2a}, R^{2b}, R^{2c}, R^{2d}, R^{2e}, and R^{2f} at each occurrence are each independently selected from the group consisting of hydrogen, C₁₋₆ optionally substituted alkyl, C₁₋₆ optionally substituted branched alkyl, C₃₋₇ optionally substituted cycloalkyl, C₁₋₆ optionally substituted haloalkyl, C₁₋₆ optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h} R³ⁱ, R^{3j}, R^{3k}, R^{3l}, and R^{3m} at each occurrence are each independently selected from the group consisting of hydrogen, C₁₋₆ optionally substituted alkyl, C₁₋₆ optionally substituted branched alkyl, C₁₋₆ optionally substituted haloalkyl, C₁₋₆ optionally substituted alkoxy, and CO₂R⁸;
R^{4a}, R^{4b}, and R^{4c} at each occurrence are each independently selected from the group consisting of hydrogen, C₁₋₆ optionally substituted alkyl, C₁₋₆ optionally substituted branched alkyl, C₁₋₆ optionally substituted cycloalkyl, C₁₋₆ optionally substituted haloalkyl, C₁₋₆ optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence is independently selected from the group consisting of hydrogen and optionally substituted alkyl;
R⁶ at each occurrence is independently selected from the group consisting of hydrogen and optionally substituted alkyl;
R⁷ at each occurrence is independently selected from the group consisting of hydrogen and optionally substituted alkyl; and
R⁸ at each occurrence is independently selected from the group consisting of hydrogen and optionally substituted alkyl.

In certain embodiments, the photodynamic compound is administered in combination with transferrin.

In certain embodiments, the photodynamic compound is administered in combination with transferrin, and the immunotherapeutic agent is at least one member selected from the group consisting of: idecabtagene vicleucel, lisocabtagene maraleucel, tisagenlecleucel, brexucabtagene autoleucel, axicabtagene ciloleucel, ciltacabtagene autoleucel, atezolizumab, avelumab, dostarlizumab, durvalumab, ipilimumab, nivolumab, pembrolizumab, pucotenlimab, pidilizumab, cemiplimab, spartalizumab, camrelizumab, cetrelimab, toripalimab, sintilimab, sasanlimab, dostarlizumab, SHR-1210, PDR001, MGA012, PDR001, AB122, AMP-224, JTX-4014, BGB-108, BCD-100, BAT1306, LZM009, AK105, HLX10, bevacizumab, trastuzumab, onartuzumab, erdafitinib, enapotamab, daratumumab, FAZ053, KN035, CS1001, SHR-1316, CBT-502, A167, STI-A101, CK-301, BGB-A333, MSB-2311, HLX20, LY3300054, sipuleucel-T, Bacillus Calmette-Guérin vaccine, nadofaragene firadonevec and T-VEC.

In certain embodiments, the photodynamic compound is administered in combination with transferrin, and the photodynamic compound is represented by formula (I) or formula (II).

In certain embodiments, the photodynamic compound is at least one member selected from the group consisting of:
Ru(2,2′-bipyridine)₂(2-(2′,2″:5″,2′″-terthiophene)-imidazo[4,5-f][1,10]phenanthroline);
Ru(2,2′-bipyridine)₂(2-(2′,2″:5″,2″;5′″,2″″-quaterthiophene)-imidazo[4,5-f][1,10]phenanthroline);
Ru(4,4′-dimethyl-2,2′-bipyridine)₂(2-thiophenimidazo[4,5-f][1,10]phenanthroline);
Ru(4,4′-dimethyl-2,2′-bipyridine)₂(2-(2′,2″-bithiophene)-imidazo[4,5-f][1,10]phenanthroline);
Ru(4,4′-dimethyl-2,2′-bipyridine)₂(2-(2′,2″:5″,2′″-terthiophene)-imidazo[4,5-f][1,10]phenanthroline);
Ru(4,4′-dimethyl-2,2′-bipyridine)₂(2-(2′,2″:5″,2′″;5′″,2″″-quaterthiophene)-imidazo[4,5-f][1,10]phenanthroline);
Ru(4,4′-di-t-butyl-2,2′-bipyridine)₂(2-thiophenimidazo[4,5-f][1,10]phenanthroline);
Ru(4,4′-di-t-butyl-2,2′-bipyridine)₂(2-(2′,2″-bithiophene)-imidazo[4,5-f][1,10]phenanthroline);
Ru(4,4′-di-t-butyl-2,2′-bipyridine)₂(2-(2′,2″:5″,2″-terthiophene)-imidazo[4,5-f][1,10]phenanthroline);
Ru(4,4′-dimethoxy-2,2′-bipyridine)₂(2-(2′,2″:5″,2″-terthiophene)-imidazo[4,5-f][1,10]phenanthroline);
Ru(5,5′-dimethyl-2,2′-bipyridine)₂(2-thiophenimidazo[4,5-f][1,10]phenanthroline);
Ru(5,5′-dimethyl-2,2′-bipyridine)₂(2-(2′,2″-bithiophene)-imidazo[4,5-f][1,10]phenanthroline);
Ru(5,5′-dimethyl-2,2′-bipyridine)₂(2-(2′,2″:5″,2′″-terthiophene)-imidazo[4,5-f][1,10]phenanthroline);
Ru(6,6′-dimethyl-2,2′-bipyridine)₂(2-thiophenimidazo[4,5-f][1,10]phenanthroline);
Ru(6,6′-dimethyl-2,2′-bipyridine)₂(2-(2′,2″-bithiophene)-imidazo[4,5-f][1,10]phenanthroline);
Ru(6,6′-dimethyl-2,2′-bipyridine)₂(2-(2′,2″:5″,2″″-terthiophene)-imidazo[4,5-f][1,10]phenanthroline);
Ru(4,4′-di(methylcarboxy)-2,2′-bipyridine)₂(2-(2′,2″:5″,2′″-terthiophene)-imidazo[4,5-f][1,10]phenanthroline);
Ru(2,2′-bipyrimidine)₂(2-(2′,2″:5″,2′″-terthiophene)-imidazo[4,5-f][1,10]phenanthroline);
Ru(2,2′-bipyrimidine)(4,4′-dimethyl-2,2′-bipyridine)(2-(2′,2″:5″,2′″-terthiophene)-imidazo[4,5-f][1,10]phenanthroline);
Ru(1,10-phenanthroline)₂(2-thiophenimidazo[4,5-f][1,10]phenanthroline);
Os(2,2′-bipyridine)₂(2-thiophenimidazo[4,5-f][1,10]phenanthroline);
Os(2,2′-bipyridine)₂(2-(2′,2″-bithiophene)-imidazo[4,5-f][1,10]phenanthroline);
Os(2,2′-bipyridine)₂(2-(2′,2″:5″,2′″-terthiophene)-imidazo[4,5-f][1,10]phenanthroline);
Os(1,10-phenanthroline)₂(2-thiophenimidazo[4,5-f][1,10]phenanthroline);
Os(1,10-phenanthroline)₂(2-(2′,2″-bithiophene)-imidazo[4,5-f][1,10]phenanthroline);
Os(1,10-phenanthroline)₂(2-(2′,2″:5″,2′″-terthiophene)-imidazo[4,5-f][1,10]phenanthroline);
Os(4,4′-dimethyl-2,2′-bipyridine)₂(2-thiophenimidazo[4,5-f][1,10]phenanthroline);
Os(4,4′-dimethyl-2,2′-bipyridine)₂(2-(2′,2″-bithiophene)-imidazo[4,5-f][1,10]phenanthroline);
Os(4,4′-dimethyl-2,2′-bipyridine)₂(2-(2′,2″:5″,2″″-terthiophene)-imidazo[4,5-f][1,10]phenanthroline); and
pharmaceutically acceptable salts thereof, and the photodynamic compound is preferably administered in combination with transferrin.

In certain embodiments, the photodynamic compound is Ru(4,4′-dimethyl-2,2′-bipyridine)₂(2-(2′,2″:5″,2′″-terthiophene)-imidazo[4,5-f][1,10]phenanthroline) or a pharmaceutically acceptable salt thereof. The photodynamic compound is preferably administered with transferrin.

In certain embodiments, the radiation is infrared light, visible light, X-rays or other ionizing radiation.

In certain embodiments, the immunotherapeutic agent, the photodynamic compound and the radiation are administered in amounts synergistically effective to treat the condition.

In certain embodiments, the immunotherapeutic agent and the photodynamic compound are independently administered to the patient locally, intravesically, intratumorally or intravenously in an amount that ranges from a Maximum Recommended Starting Dose based on a Human Equivalent Dose to a Therapeutic Dose or a Biologically Effective Dose.

In certain embodiments, step (a) is conducted before step (b) and step (b) is conducted before step (c).

In certain embodiments, the cancer is characterized by expression of PD-1, PD-L1 or PD-L2.

In certain embodiments, the cancer is selected from the group consisting of: bone cancer, pancreatic cancer, skin cancer, brain cancer, lung cancer, colorectal cancer, head cancer, neck cancer, cutaneous malignant melanoma, intraocular malignant melanoma, uterine cancer, ovarian cancer, rectal cancer, anal cancer, stomach cancer, testicular cancer, uterine cancer, fallopian tube carcinoma, carcinoma of the endometrium, endometrial cancer, carcinoma of the cervix, carcinoma of the vagina, carcinoma of the vulva, Hodgkin's Disease, non-Hodgkin's lymphoma, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, leukemia, solid tumors of childhood, lymphocytic lymphoma, cancer of the bladder, cancer of the kidney or urethra, carcinoma of the renal pelvis, neoplasm of the Central Nervous System (“CNS”), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancer, and combinations thereof.

These and other objects, features and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference. The citation of any document is not to be construed as an admission that it is prior art with respect to the present invention.

### BRIEF DESCRIPTION OF SEVERAL VIEWS OF THE DRAWINGS

The patent or application file contains at least one drawing executed in color. Copies of this patent or patent application publication with color drawing(s) will be provided by the Office upon request and payment of the necessary fee.

The invention will be described in conjunction with the following drawings, wherein:

FIG. 1A is a bar chart of percent change in CD47 expression in U87 human glioma cells;

FIG. 1B is a bar chart of percent change in CD47 expression in T24 human urinary bladder cancer cells;

FIG. 2 is a bar chart of percentage of cell surface calreticulin;

FIG. 3 is a bar chart of extracellular ATP concentration;

FIG. 4 is a bar chart of anti-tumor immunity gene expression of a control and four different test samples;

FIG. 5A is a photograph of Rat Glioma (“RG2”) cells at 4 times magnification;

FIG. 5B is a photograph of RG2 at 20 times magnification;

FIG. 5C is a bar chart showing T cell count per square meter of tumor;

FIG. 5D is a photograph of RG2 at 4 times magnification;

FIG. 5E is a photograph of RG2 at 20 times magnification;

FIG. 5F is a photograph of RG2 at 4 times magnification;

FIG. 5G is a photograph of RG2 at 20 times magnification;

FIG. 6 is a bar chart showing percentage change in CD47 expression;

FIG. 7 is a bar chart showing cell surface calreticulin percentage;

FIG. 8 is a bar chart showing ATP concentration; and

FIG. 9 is a photograph showing intracellular localization of TLD-1433 in cancer cells.

### DETAILED DESCRIPTION OF PREFERRED EMBODIMENTS OF THE INVENTION

### Glossary

Throughout the description, where compositions are described as: having, including or comprising specific components or where processes are described as: having, including or comprising specific process steps, it is contemplated that compositions of the present teachings also consist essentially of, or consist of, the recited components and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

For the purposes of the present invention the terms “compound,” “analog,” and “composition of matter” stand equally well for the inventive compounds described herein, be they photodynamic or not, including all enantiomeric forms, diastereomeric forms, salts and the like and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to: diastereomeric salt formation, kinetic resolution and asymmetric synthesis. The present teachings also encompass cis and trans isomers of compounds containing alkenyl moieties (e.g.: alkenes and imines). It is also understood that the present teachings encompass all possible regioisomers, and mixtures thereof, which can be obtained in pure form by standard separation procedures known to those skilled in the art, and include, but are not limited to: column chromatography, thin-layer chromatography and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include: metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g.: ethyl-tert-butyl-, diethyl-, diisopropyl-, triethyl-, tributyl- or dimethylpropylamine), or a mono-, di-, or trihydroxy lower alkylamine (e.g.: mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic, as well as other known pharmaceutically acceptable acids.

When any variable occurs more than one time in any constituent or in any formula, its definition in each occurrence is independent of its definition at every other occurrence (e.g.: in N(R⁶)₂, each R⁶ may be the same or different than the other). Combinations of substituents and/or variables are permissible only if such combinations result in stable compounds.

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

As used herein, the term “photodynamic therapy” or “PDT” refers to a treatment for destroying cells or modulating immune function, including immune response, of cells and tissue through use of a drug that can be activated by light of a certain wavelength and dose.

As used herein, the term “radiation therapy” or “RT” refers to a treatment for destroying cells or modulating immune function, including immune response, of cells and tissue through use of a drug that can be activated by ionizing radiation of a certain wavelength and dose.

As used herein, the term “photodynamic compound” or “PDC” refers to a compound that can be activated by light of a certain wavelength and dose for PDT. The term is also used herein to refer to a compound that can be activated by ionizing radiation of a certain wavelength and dose for RT.

As used herein, the term “radiation” used without the term “ionizing” is intended to encompass all types of radiation in the electromagnetic spectrum including light and ionizing radiation.

As used herein, the term “immunotherapy” refers to a treatment which elicits an immune response from a patient so as to prevent, ameliorate or cure a condition (e.g., a disease or an infection).

An “immunotherapeutic agent” is a substance that elicits an immune response from a patient, so as to prevent, ameliorate or cure a condition (e.g.: a disease or an infection).

Except when noted, the terms “subject” or “patient” are used interchangeably and refer to mammals such as human patients and non-human primates, as well as experimental animals such as: dogs, rabbits, rats, mice and other animals. Accordingly, the term “subject” or “patient” as used herein means any mammalian patient or subject to which the compounds of the invention can be administered. In an exemplary embodiment of the present invention, to identify subjects or patients for treatment according to the methods of the invention, accepted screening methods are employed to determine risk factors associated with a targeted or suspected disease or condition or to determine the status of an existing disease or condition in a subject or patient. These screening methods include, for example: conventional work-ups to determine risk factors that may be associated with the targeted or suspected disease or condition. These and other routine methods allow the clinician to select patients in need of therapy using the methods and compounds of the present invention.

As used herein, the expression “biological target” refers to an organ, tissue and/or cell of an organism and/or to the organism itself.

As used herein the term “immunogenic” refers to a substance that is able to elicit an immune response.

The term “substituted” means that an atom or group of atoms formally replaces hydrogen as a “substituent” attached to another group. The term “substituted”, unless otherwise indicated, refers to any level of substitution, e.g.: mono-, di-, tri-, tetra- or penta-substitution, where such substitution is permitted. The substituents are independently selected and substitution may be at any chemically accessible position. It is to be understood that substitution at a given atom is limited by valency. It is to be understood that substitution at a given atom results in a chemically stable molecule. The phrase “optionally substituted” means unsubstituted or substituted. A single divalent substituent, e.g.: oxo, can replace two hydrogen atoms.

The term “alkyl” employed alone or in combination with other terms, refers to a saturated hydrocarbon group that may be straight-chained or branched. Examples of alkyl moieties include, but are not limited to: chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. Examples of alkenyl groups include, but are not limited to: ethenyl, n-propenyl, isopropenyl, n-butenyl, sec-butenyl and the like.

The term “alkoxy”, employed alone or in combination with other terms, refers to a group of formula-O-alkyl, wherein the alkyl group is as defined above. Examples of alkoxy groups include: methoxy, ethoxy, propoxy (e.g.: n-propoxy and isopropoxy), t-butoxy and the like.

All compounds, and pharmaceutically acceptable salts thereof, can be found together with other substances such as water and solvents (e.g.: hydrates and solvates) or can be isolated. When in the solid state, the compounds described herein and salts thereof may occur in various forms and may, for example, take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the invention, or salts thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive: toxicity, irritation, allergic response, or other problem or complication, commensurate with a reasonable benefit/risk ratio.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to: mineral or organic acid salts of basic residues such as amines; alkali or organic salts of acidic residues such as carboxylic acids; and the like. The pharmaceutically acceptable salts of the present invention include the non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like: ether, ethyl acetate, alcohols (e.g.: methanol, ethanol, iso-propanol or butanol) or acetonitrile (MeCN) are preferred. Lists of suitable salts are found in Remington's Pharmaceutical Sciences, 17.sup.th Ed., (Mack Publishing Company, Easton, 1985), p. 1418, Berge et al., J. Pharm. Sci., 1977, 66 (1), 1-19 and in Stahl et al., Handbook of Pharmaceutical Salts: Properties, Selection and Use, (Wiley, 2002). In some embodiments, the compounds described herein include the N-oxide forms.

### Therapeutic Method

The method of the invention comprises administering to a patient (i.e.: an animal, and preferably a mammal, such as a human) an effective amount of at least one immunotherapeutic agent and at least one PDC (sometimes collectively referred to herein as “active agents”) and administering light and/or ionizing radiation to the patient so as to treat a condition associated with hyperproliferating cells.

The sequence in which the active agents and radiation (i.e., light and/or ionizing radiation) are administered to the patient can be varied for optimal results. They can be administered in any order, separately or together. In one preferred embodiment, the immunotherapeutic agent is administered before the PDC is administered and the radiation is administered after the PDC is administered. In another preferred embodiment, the PDC is administered before the radiation is administered and the immunotherapeutic agent is administered after the radiation is administered.

Each of the active agents is preferably administered in a composition further comprising a pharmaceutically acceptable excipient. In certain embodiments, the PDC and the immunotherapeutic agent are in separate compositions, while in other embodiments, they are in the same composition.

For the purposes of the present invention the terms “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable and functional pharmaceutical, serving not only as part of the overall vehicle for delivery, but also as a means for achieving effective absorption by the recipient of the active ingredient. An excipient may fill a role as simple and direct as being an inert filler, or an excipient as used herein may, for example, be part of a pH stabilizing system or coating to insure delivery of the ingredients safely to the tissue.

Examples of such excipients or carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, PA (1985), the entire disclosure of which is incorporated by reference herein for all purposes. Supplementary active ingredients can also be incorporated into the pharmaceutical compositions.

Active agents of the invention can be administered: orally, intravenously, intravesically, lingually, intratumorally, topically or parenterally, neat or in combination with conventional pharmaceutical carriers. Applicable solid carriers can include one or more substances which can also act as: flavoring agents, lubricants, solubilizers, suspending agents, fillers, glidants, compression aids, binders or tablet-disintegrating agents or encapsulating materials. The active agents can be formulated in a conventional manner, for example, in a manner similar to that used for known active agents. Oral formulations containing an active agent disclosed herein can comprise any conventionally used oral form, including: tablets, capsules, buccal forms, troches, lozenges and oral liquids, suspensions or solutions. In powders, the carrier can be a finely divided solid, which is an admixture with a finely divided active agent. In tablets, an active agent disclosed herein can be mixed with a carrier having the necessary compression properties in suitable proportions and compacted in the shape and size desired. The powders and tablets can contain up to 99% of the active agent.

Capsules can contain mixtures of one or more compound(s) and/or compositions disclosed herein with inert filler(s) and/or diluent(s) such as: pharmaceutically acceptable starches (i.e.: corn, potato or tapioca starch), sugars, artificial sweetening agents, powdered celluloses (i.e.: crystalline and microcrystalline celluloses), flours, gelatins, gums and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to: magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes and ion exchange resins. Surface modifying agents include nonionic and anionic surface modifying agents. Representative examples of surface modifying agents include, but are not limited to: poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate and triethanolamine. Oral formulations herein can utilize standard delay or time-release formulations to alter the absorption of the compound(s) and/or compositions. The oral formulation can also consist of administering an active agent disclosed herein in water or fruit juice, containing appropriate solubilizers or emulsifiers, as needed.

Liquid carriers can be used in preparing solutions, suspensions, emulsions, syrups, elixirs and for inhaled delivery. An active agent of the invention can be dissolved or suspended in a pharmaceutically acceptable liquid carrier such as: water, an organic solvent, a mixture of both or a pharmaceutically acceptable oil or fat. The liquid carrier can contain other suitable pharmaceutical additives such as: solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to: water (particularly containing additives as described herein, for example, cellulose derivatives such as a sodium carboxymethyl cellulose solution), alcohols (including monohydric alcohols and polyhydric alcohols, for example, glycols) and their derivatives, and oils (i.e.: fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example: intramuscular, intraperitoneal, topical or subcutaneous injection. Sterile solutions can also be administered intravenously. Compositions for oral administration can be in either liquid or solid form.

In certain embodiments, the pharmaceutical composition is in unit dosage form, for example as: tablets, capsules, powders, solutions, suspensions, emulsions, granules or suppositories. In such form, the pharmaceutical composition can be sub-divided into unit dose(s) containing appropriate quantities of the active agent. The unit dosage forms can be packaged compositions, for example: packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of each active agent to about 500 mg/kg of each active agent and can be given in a single dose or in two or more doses. Such doses can be administered in any manner useful in directing the compound(s) and/or composition(s) to the target tissue and/or bloodstream of the patient, including: orally, via implants, parenterally (including intravenous, intraperitoneal, topical and subcutaneous injections), rectally, vaginally and transdermally.

When administered for the treatment or inhibition of a particular disease state or disorder, it is understood that an effective dosage can vary depending upon the particular active agent utilized, the mode of administration and severity of the condition being treated, as well as the various physical factors related to the individual being treated. In therapeutic applications, an active agent can be provided to a patient already suffering from a disease in an amount sufficient to heal or at least partially ameliorate the symptoms of the disease and its complications. The dosage to be used in the treatment of a specific patient typically must be subjectively determined by the attending physician. The variables involved include the specific condition and its state as well as the: physical size, age, gender, health status and response pattern of the patient.

In some cases, it may be desirable to administer the active agents directly to the airways of the patient, using devices such as, but not limited to: metered dose inhalers, breath-operated inhalers, multidose dry-powder inhalers, pumps, squeeze-actuated nebulized spray dispensers, aerosol dispensers and aerosol nebulizers. For administration by intranasal or intrabronchial inhalation, the active agent(s) can be formulated into a liquid composition, a solid composition, or an aerosol composition. The liquid composition can include, by way of illustration, one or more active agents dissolved, partially dissolved or suspended in one or more pharmaceutically acceptable solvents and can be administered by, for example, a pump or a squeeze-actuated nebulized spray dispenser. The solvents can be administered by, for example, isotonic saline or bacteriostatic water. The solid composition can be administered, by way of illustration, a powder preparation including one or more active agents intermixed with lactose or other inert powders that are acceptable for intrabronchial use, and can be administered by, for example, an aerosol dispenser or a device that breaks or punctures a capsule encasing the solid active agent and delivers the solid active agent for inhalation. The aerosol active agent can include, by way of illustration, one or more active agents, propellants, surfactants and co-solvents, and can be administered by, for example, a metered device. The propellants can be a chlorofluorocarbon, a hydrofluoroalkane, or other propellants that are physiologically and environmentally acceptable.

The active agents of the invention can be administered parenterally or intraperitoneally. Solutions or suspensions of these active agents or pharmaceutically acceptable salts, hydrates, or esters thereof can be prepared in water suitably mixed with a surfactant such as hydroxylpropylcellulose. Dispersions can also be prepared in propylene glycol, glycerol, liquid polyethylene glycols and/or mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In certain embodiments, the form can be sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example: water, ethanol, polyol (i.e.: propylene glycol, glycerol and liquid polyethylene glycol) and/or suitable mixtures thereof in oils.

Active agents described herein can be administered transdermally (i.e.: administered across the surface of the body and the inner linings of bodily passages including epithelial and mucosal tissues). Such administration can be carried out using the active agents of the invention including pharmaceutically acceptable: salts, hydrates, or esters thereof, in lotions, creams, foams, patches, suspensions, solutions and/or suppositories (rectal and vaginal).

Transdermal administration can be accomplished through the use of a transdermal patch containing an active agent disclosed herein, and a carrier that can be inert to the active agent, can be non-toxic to the skin and can allow delivery of the active agent for systemic absorption into the blood stream via the skin. The carrier can take any number of forms such as creams and ointments, pastes, gels and occlusive devices. The creams and ointments can be viscous liquid or semisolid emulsions of either the oil-in-water or water-in-oil type. Pastes comprised of absorptive powders dispersed in petroleum or hydrophilic petroleum containing the active agent can also be suitable. A variety of occlusive devices can be used to release the active agent into the blood stream, such as a semi-permeable membrane covering a reservoir containing the active agent with or without a carrier, or a matrix containing the active agent. Other occlusive devices are known in the literature.

Compounds and/or compositions described herein can be administered rectally or vaginally in the form of a conventional suppository. Suppository formulations can be made from traditional materials, including cocoa butter, with or without the addition of waxes to alter the suppository's melting point and/or glycerin. Water-soluble suppository bases, such as polyethylene glycols of various molecular weights, can also be used.

Lipid formulations or nanocapsules can be used to introduce active agents into host cells either in vitro or in vivo. Lipid formulations and nanocapsules can be prepared by methods known in the art.

To increase the effectiveness of active agents, it can be desirable to combine an active agent with other agents effective in the treatment of the target disease. For example, other active agents effective in treating the target disease can be administered with the active agents. The other agents can be administered at the same time or at different times than the active agents disclosed herein.

Active agents of the invention can be useful for the treatment or inhibition of a pathological condition or disorder in a mammal, for example, a human patient. The invention accordingly provides methods of treating or inhibiting a pathological condition or disorder by providing to a mammal an active agent of the invention.

In certain embodiments, the inventive method is effective to inhibit the activity of PD-1/PD-L1 protein/protein interaction and, thus, is useful in treating diseases and disorders associated with activity of PD-1 and the diseases and disorders associated with PD-L1; including, its interaction with other proteins such as PD-1 and B7-1 (CD80). In certain embodiments, the method is useful for therapeutic administration to enhance, stimulate and/or increase immunity in cancer, chronic infection or sepsis, including enhancement of response to vaccination.

In certain embodiments, the method is effective to treat a condition associated with hyperproliferating cells, which typically results in a benign or malignant tumor. The method is particularly suitable for treating cancer.

Examples of cancers that are treatable by the method of the invention include, but are not limited to: bone cancer, pancreatic cancer, skin cancer, brain cancer, lung cancer, colorectal cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the Central Nervous System (“CNS”), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers, especially metastatic cancers that express PD-L1.

Preferred embodiments of the inventive method are synergistically effective for treating conditions associated with hyperproliferating cells, such as benign and malignant tumors, and for treating conditions associated with activity of PD-1 and/or PD-L1.

The dosages of the active agents can vary according to, for example, the particular use for which the treatment is made, the manner of administration of the compound, the health and condition of the patient and the judgment of the prescribing physician. The proportion or concentration of a compound of the invention in a pharmaceutical composition can vary depending upon a number of factors including: dosage, chemical characteristics (e.g.: hydrophobicity) and the route of administration. For example, the PDC and the immunotherapeutic agent can each be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. In certain embodiments, the dosage form comprises each active agent in amounts from about 0.001 mg to about 1000 mg or 0.01 mg to 100 mg or 0.1 mg to 10 mg. The dosage is likely to depend on such variables as the type and extent of progression of the disease or disorder, the overall health status of the particular patient, the relative biological efficacy of the compound selected, formulation of the excipient and its route of administration. Effective doses can be extrapolated from dose-response curves derived from in vitro or in vivo model test systems. Preferably, the immunotherapeutic agent is administered in an anti-tumor effective amount when used to treat cancer.

In certain embodiments, the immunotherapeutic agent will be administered systemically either prior to or after the PDC administration (treatments vary from a few months to a few years). In certain embodiments, the immunotherapeutic agent is administered to the patient: locally, intravesically, intratumorally, intravenously or by other administration methods: daily, every 2 days, 3 times per week, every 3 days, weekly, biweekly, monthly, quarterly or some other administration schedule based on the patient's disease, stage and grade in an amount that ranges from the Maximum Recommended Starting Dose (“MRSD”) based on the Human Equivalent Dose (“HED”) to the Therapeutic Dose (“TD”) or Biologically Effective Dose (“BED”).

In certain embodiments, the PDC will be administered locally and light activated (1 to 3 treatments) or the PDC will be administered systemically and activated via radiation (3 treatments per week over 2 to 6 weeks, with radiation daily Monday to Friday for the same time period). In certain embodiments, the PDC is administered to the patient: locally, intravesically, intratumorally, intravenously or by other administration methods: daily, every 2 days, 3 times per week, every 3 days, weekly, biweekly, monthly, quarterly or some other administration schedule based on the patient's disease, stage and grade in an amount that ranges from the MRSD based on the HED to the TD or BED.

Radiation Therapy is administered to the patient to activate at least one PDC present to treat the condition. The term “radiation” as used herein encompasses non-ionizing radiation and ionizing radiation of the electromagnetic spectrum, including: infrared light, visible light, X-rays, Y-rays and quanta, and corpuscular radiation (i.e.: a-particles, p-particles, positrons, neutrons and heavy particles) capable of producing ions, in the less than 180 nanometers (“nm”) range. Suitable wavelengths of light activation; include, but are not limited to 180 to 1000 nm and most preferably 400 to 950 nm.

Radiation is directly ionizing if it carries an electric charge that directly interacts with atoms in the tissue or medium by electrostatic attraction. Indirect ionizing radiation is not electrically charged, but results in production of charged particles by which its energy is absorbed. It takes about 34 eV of energy to produce an ionization. Most human exposures to radiation are of energies of 0.05-5 Million electron Volts (MeV)-energies at which many ionizations occur as the radiation passes through cells. Most X-rays have a wavelength ranging from 0.001 to 10 nanometers. In the case of using a radio enhancer, a patient can be treated with a “diagnostic” dose of ionizing radiation, such as 0.02 Gray (“Gy”).

The radiation can be applied systemically or locally, topically or internally. The radiation is administered in a safe and effective dosage. For example, laser light is preferably administered in a dosage of at least 10 J/cm², preferably 10 or 100 J/cm² and more preferably from 25 to 90 J/cm². Radiation is preferably administered at a predetermined fluence rate or radiation dose to achieve the most desirable therapeutic effect-up to the highest permissible radiation dose, based on the patient's clinical status.

### Immunotherapeutic Agents

The invention comprises the use of at least one immunotherapeutic agent in combination with at least one PDC. The immunotherapeutic agent and the PDC can be administered together or separately in any order.

Preferred immunotherapeutic agents include: CAR-T cell therapeutic agents; including, but not limited to: idecabtagene vicleucel, lisocabtagene maraleucel, tisagenlecleucel, brexucabtagene autoleucel, axicabtagene ciloleucel and ciltacabtagene autoleucel.

Other preferred immunotherapeutic agents include MABs designed to downregulate or block PD-1, PD-L1 and/or PD-L2.

Suitable anti-PD-1 monoclonal antibodies: include, but are not limited to: nivolumab, pembrolizumab (also known as MK-3475), pucotenlimab, pidilizumab, cemiplimab, spartalizumab, camrelizumab, cetrelimab, toripalimab, sintilimab, sasanlimab, dostarlizumab, SHR-1210, PDR001, MGA012, PDR001, AB122, AMP-224, JTX-4014, BGB-108, BCD-100, BAT1306, LZM009, AK105 and HLX10.

Suitable anti-PD-L1 monoclonal antibodies include but are not limited to BMS-935559, MEDI4736, MPDL3280A (also known as RG7446), durvalumab, atezolizumab, avelumab, MSB0010718C, tislelizumab, bevacizumab, trastuzumab, onartuzumab, erdafitinib, enapotamab, daratumumab, FAZ053, KN035, CS1001, SHR-1316, CBT-502, A167, STI-A101, CK-301, BGB-A333, MSB-2311, HLX20 or LY3300054.

Suitable anti-PD-L2 monoclonal antibodies; include, but are not limited to: IO120.

Recently, a number of PD-L1 (such as IO103) or PD-L2 (such as IO120) peptides, which can activate specific T cells inducing anti-regulatory functions including cytotoxicity against PD-L1/PD-L2-expressing cells has been introduced to the clinical development. (Klausen et al. “An immunogenic first-in-human immune modulatory vaccine with PD-L1 and PD-L2 peptides is feasible and shows early signs of efficacy in follicular lymphoma.” OncoImmunology 10, no. 1 (2021): 1975889.)

Moreover, Pre-clinical studies demonstrated that dual blockade of both VISTA and PD-L1 can be synergistic (Liu et al. “Immune-checkpoint proteins VISTA and PD-1 nonredundantly regulate murine T-cell responses.” Proceedings of the National Academy of Sciences 112, no. 21 (2015): 6682-6687.) CA-170 is a first-in-class small molecule oral inhibitor that directly targets VISTA and PDL1/L2 and has demonstrated anti-tumor activity in multiple preclinical models. This presentation is an update to the on-going Phase 1 trial (Clinicaltrials.org NCT02812875) [Radhakrishnan V S, et al., Poster P714, SITC 2018].

Blocking both PD-L1 and PD-L2 could more fully restore tumor-specific T cell activation and potentiate anti-cancer immunotherapy. In vivo, ADCC-capable PD-Ligand bispecific antibodies suppress the growth of U2940 lymphoma in immunodeficient mice more efficiently than Rituximab, and in a syngeneic model of PD-L1/PD-L2 double positive colon carcinoma, these antibodies demonstrate superiority to PD-1 blocking antibodies to limit tumor growth and increase survival. Furthermore, treatment with bispecific antibodies increases T cell proliferation and cytotoxicity and reduces density of immunosuppressive myeloid stroma in vivo (Couillault et al. “291 Dual-specific antibodies blocking both PD-L1 and PD-L2 engagement of PD-1 restore anti-tumor immunity.” J Immunother Cancer 2021; 9 (Suppl 2): A1-A1054). Therefore, PD-L2 should be considered as crucial immunosuppressive mechanisms in clinical settings in addition to PD-L1. The Cancer Genome Atlas (TCGA) datasets revealed a strong correlation between decreased antitumor immune responses and PD-L2 rather than PD-L1 in the tumor microenvironment (TME) in several cancers, including renal cancer and lung squamous cell carcinoma (Tanegashima et al. “Immune suppression by PD-L2 against spontaneous and treatment-related antitumor immunity.” Clinical Cancer Research 25, no. 15 (2019): 4808-4819). Indeed, blocking only PD-1 or only PD-L1 or PD-L2 thus does not relieve all inhibitory components of this and additional check-point pathways should be considered as a non-optimised treatment strategy in oncology. Therefore, bispecific or even tri-specific antibodies is under development (Mertens et al. “New recombinant bi- and trispecific antibody derivatives.” Novel Frontiers in the Production of Compounds for Biomedical Use (2002): 195-208).

The instant invention enhances and optimizes the cytotoxic effector (therapeutic) function of these antibodies by PDCs through the depletion of tumor cells and supportive stroma.

Use of bispecific PD-L1/PD-L2 and/or even multi (tri-specific, etc.) antibodies in combination with PDCs that inhibit CD 47 check-point will provide an additional, optimized, check-point modulation, and hence, will more effectively restore the function of suppressed T and B cells. Moreover, human antibodies, including multi-specific antibodies in combination with PDCs could lead to significantly higher FcγRIIa and antigen presenting cells (APC) activation than FDA-approved clinical human IgG1 PD-L1 antibodies, as well as bispecific and/or trispecific antibodies, which are currently under development.

Other preferred immunotherapeutic agents include cancer vaccines; including, but not limited to: sipuleucel-T (Provenge), Bacillus Calmette-Guérin (“BCG”) vaccine, nadofaragene firadonevec (ADSTILADRIN) and T-VEC (Imlygic).

### PDCs

PDCs suitable for use in the invention; include, but are not limited to: those disclosed in WO 2013158550 A1, WO 2014145428 A2, U.S. Pat. Nos. 6,962,910, 7,612,057, 8,445,475 and 8,148,360.

Preferred PDCs are represented by one of the following formulas:
(a) formula (I):

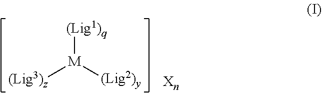

including hydrates, solvates, pharmaceutically acceptable salts, prodrugs and complexes thereof, wherein:
M at each occurrence is independently selected from the group consisting of osmium, ruthenium and rhodium;
X is selected from the group consisting of Cl⁻, PF₆⁻, Br⁻, BF₄⁻, ClO₄⁻, CF₃SO₃⁻, and SO₄⁻²;
n=0, 1, 2, 3, 4, or 5;
q is independently at each occurrence 0, 1, or 2;
y is independently at each occurrence 0, 1, or 2;
z is independently at each occurrence 1, 2, or 3;
Lig¹ is a bidentate ligand that at each occurrence is each independently selected from the group consisting of

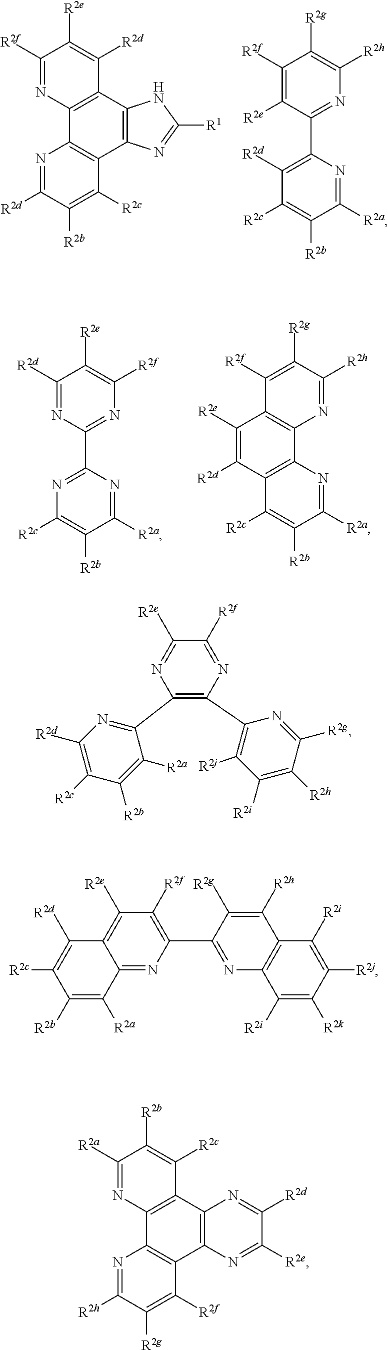

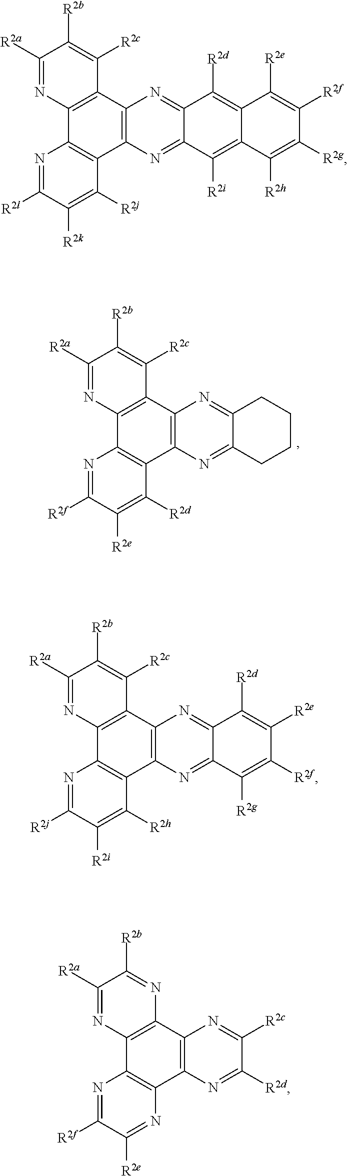

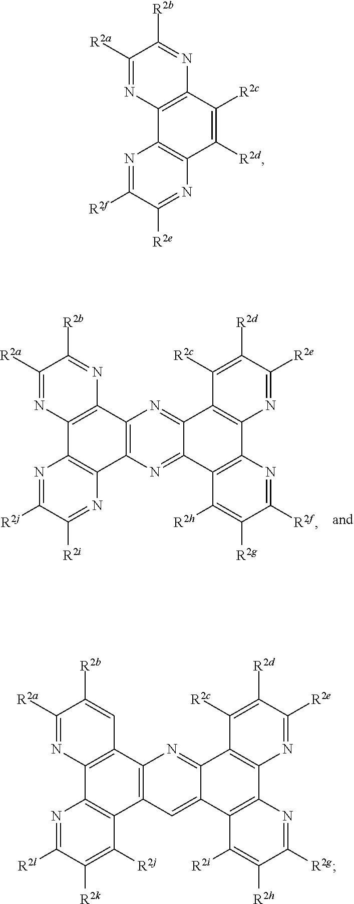

Lig² is a bidentate ligand that at each occurrence is each independently selected from the group consisting of

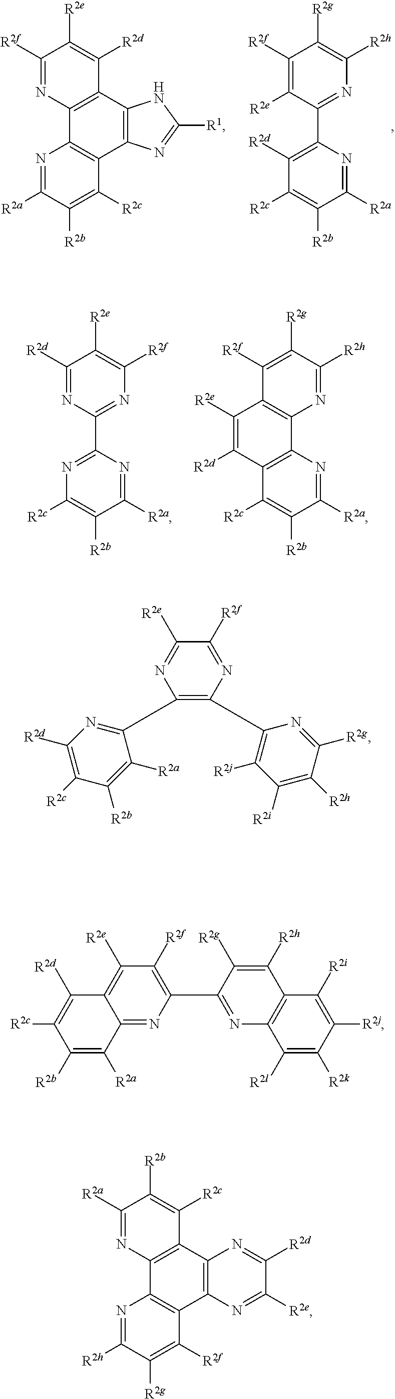

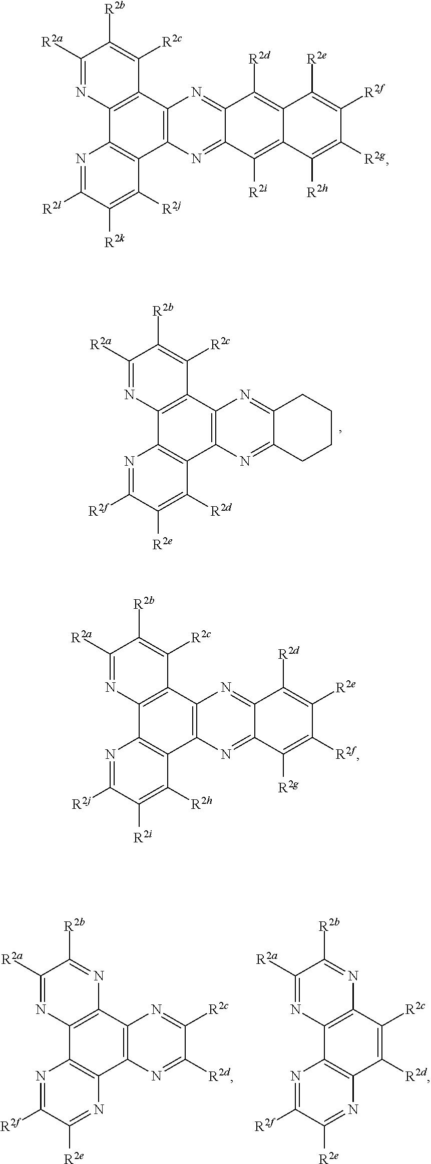

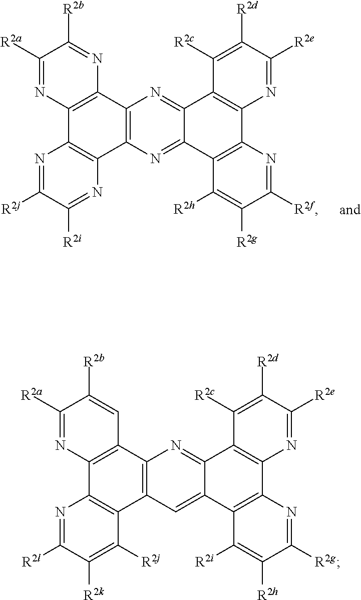

Lig³ is a bidentate ligand that at each occurrence is each independently selected from the group consisting of

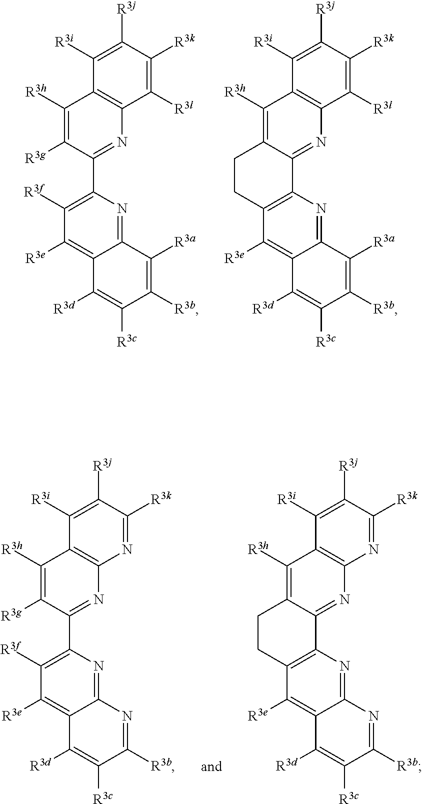

R¹ is selected from the group consisting of hydrogen, optionally substituted phenyl, optionally substituted aryl, optionally substituted heteroaryl, 4-pyridyl, 3-pyridyl, 2-thiazole, 2-pyrolyl, 2-furanyl,

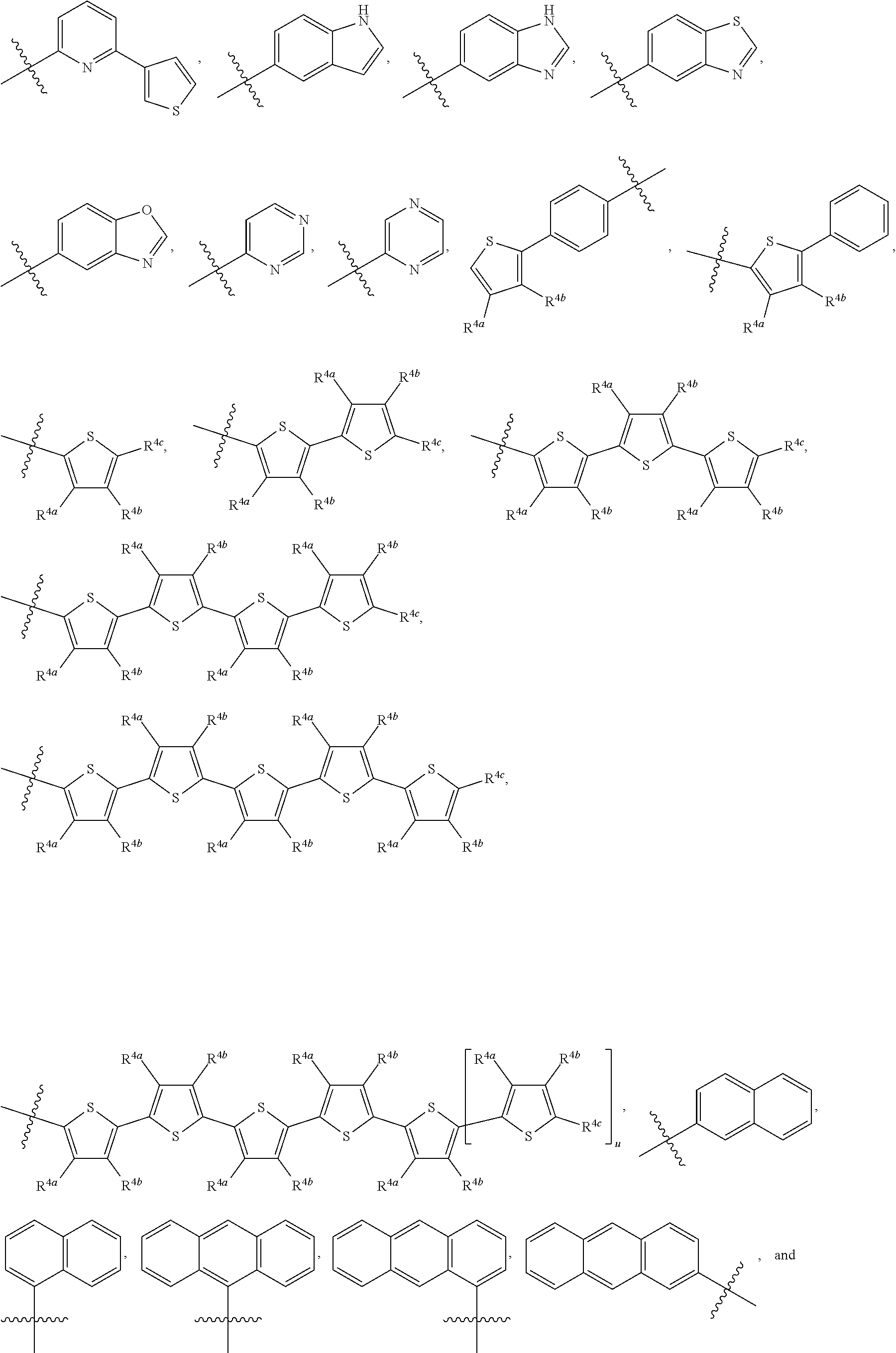

u is an integer, which is preferably 1, 2, 3, 4, 5, 6, 7, 8, 9 or 10;
R^{2a}, R^{2b}, R^{2c}, R^{2d}, R^{2e}, R^{2f}, R^{2g}, R^{2h}, R²ⁱ, R^{2j}, R^{2k}, and R^{2l} at each occurrence are each independently selected from the group consisting of hydrogen, C₁₋₆ optionally substituted alkyl, C₁₋₆ optionally substituted branched alkyl, C₃₋₇ optionally substituted cycloalkyl, C₁₋₆ optionally substituted haloalkyl, C₁₋₆ optionally substituted alkoxy, CO₂R⁵, CONR⁶₂, NR⁷₂, SO₃H, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h} R³ⁱ, R^{3j}, R^{3k}, and R^{3l} at each occurrence are each independently selected from the group consisting of hydrogen, C₁₋₆ optionally substituted alkyl, C₁₋₆ optionally substituted branched alkyl, C₁₋₆ optionally substituted haloalkyl, C₁₋₆ optionally substituted alkoxy, optionally substituted phenyl, and CO₂R⁸;
R^{4a}, R^{4b}, and R^{4c} at each occurrence are each independently selected from the group consisting of hydrogen, C₁₋₆ optionally substituted alkyl, C₁₋₆ optionally substituted branched alkyl, C₁₋₆ optionally substituted cycloalkyl, C₁₋₆ optionally substituted haloalkyl, C₁₋₆ optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence are each independently selected from the group consisting of hydrogen and optionally substituted alkyl;
R⁶ at each occurrence are each independently selected from the group consisting of hydrogen and optionally substituted alkyl;
R⁷ at each occurrence are each independently selected from the group consisting of hydrogen and optionally substituted alkyl; and
R⁸ at each occurrence are each independently selected from the group consisting of hydrogen and optionally substituted alkyl;
(b) formula (VI):

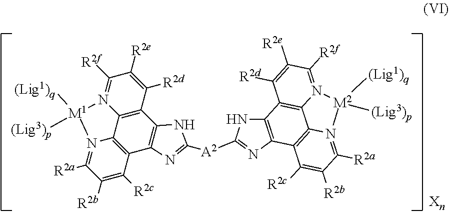

including hydrates, solvates, pharmaceutically acceptable salts, prodrugs and complexes thereof wherein;
M¹ and M² at each occurrence is independently selected from the group consisting of osmium, manganese, molybdenum, rhenium, ruthenium, iron, cobalt, rhodium, iridium, nickel, platinum, and copper;
A² is selected from the group consisting of

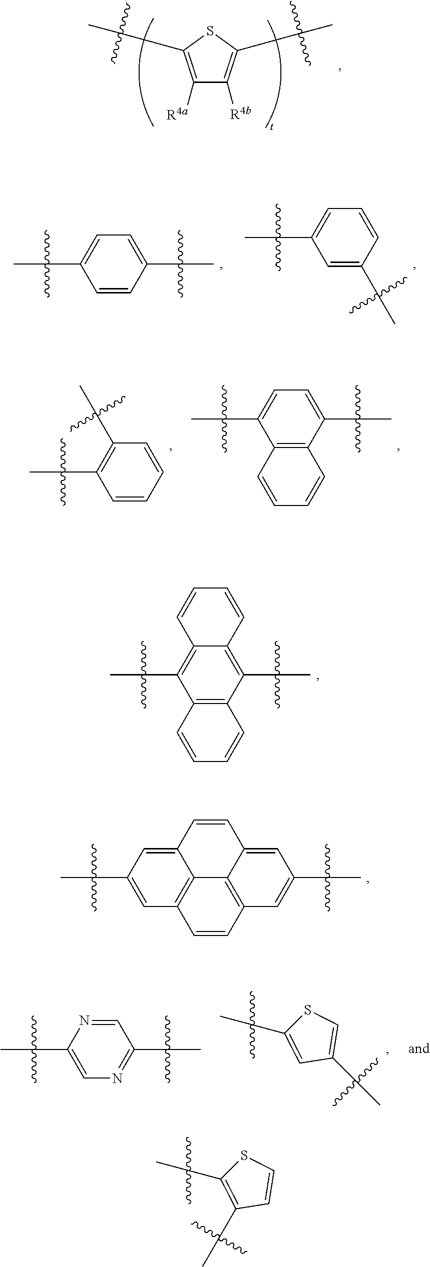

t is an integer, which is preferably 1, 2, 3, 4, 5, 6, 7, 8, 9 or 10;
(c) formula (VIIa):

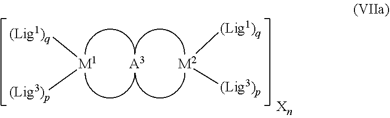

including hydrates, solvates, pharmaceutically acceptable salts, prodrugs and complexes thereof wherein:
A³ is selected from the group consisting of

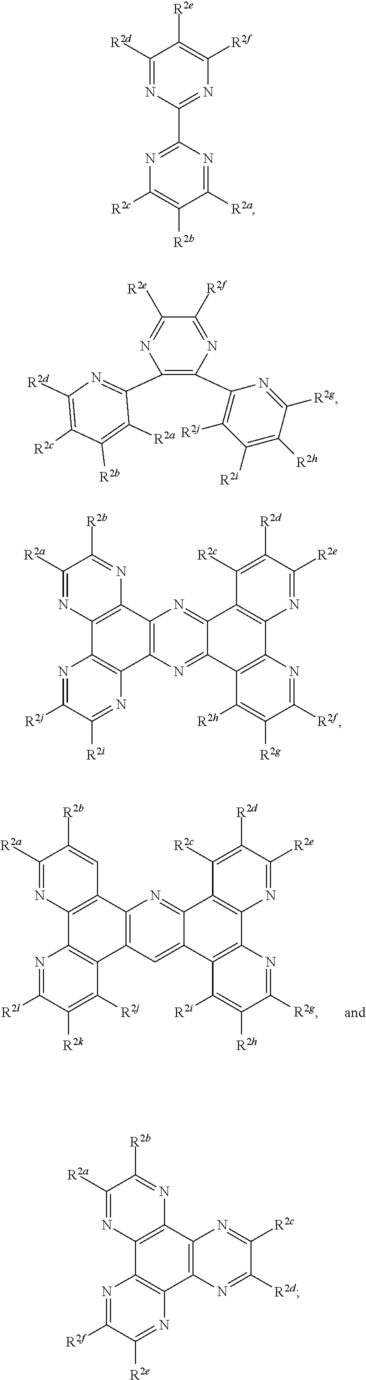

Lig¹ is a bidentate ligand that at each occurrence is each independently selected from the group consisting of

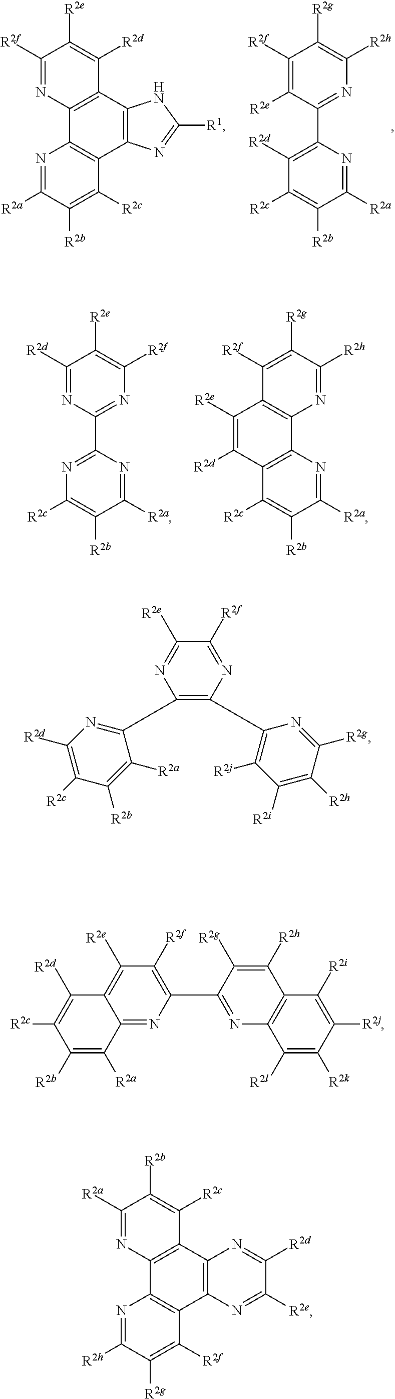

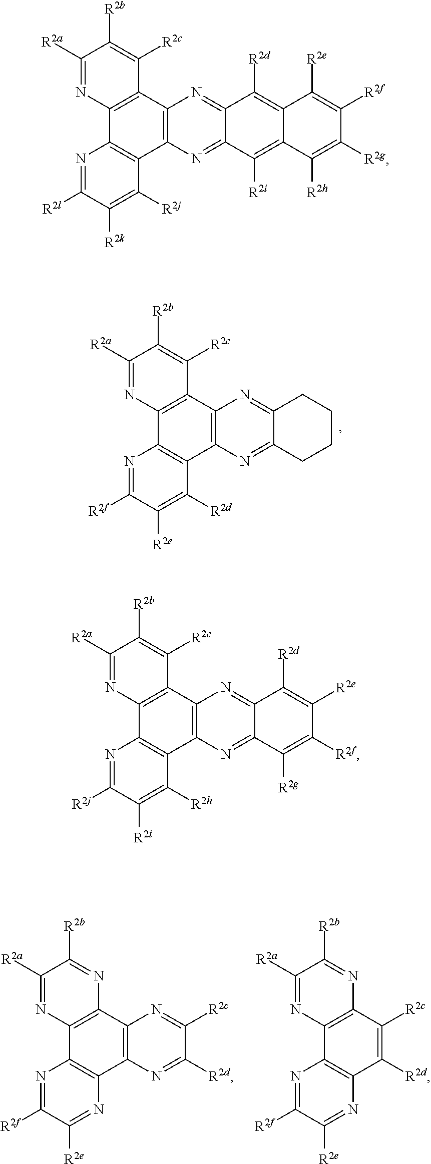

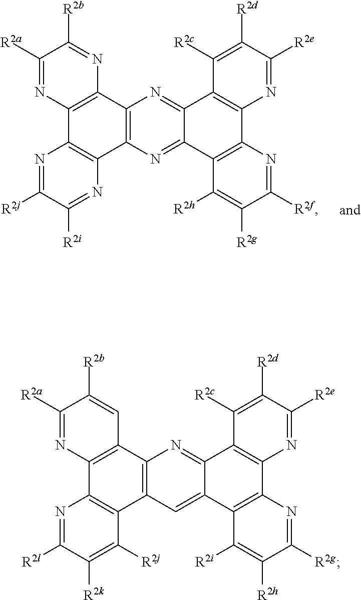

Lig³ is a bidentate ligand that at each occurrence is each independently selected from the group consisting of

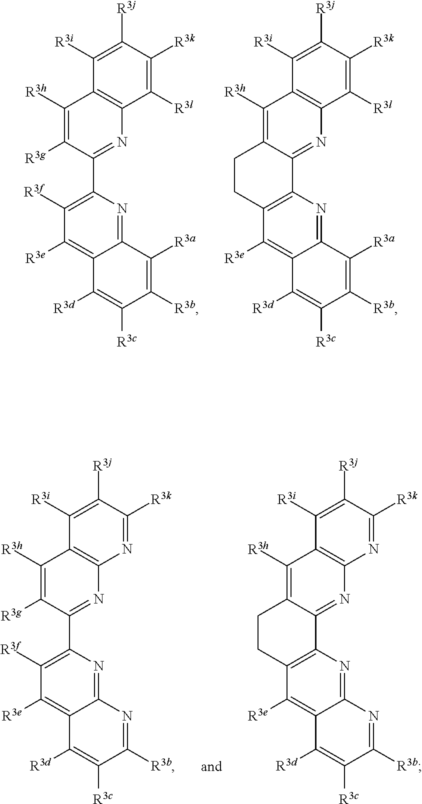

R¹ is selected from the group consisting of hydrogen, optionally substituted phenyl, optionally substituted aryl, optionally substituted heteroaryl, 4-pyridyl, 3-pyridyl, 2-thiazole, 2-pyrolyl, 2-furanyl,

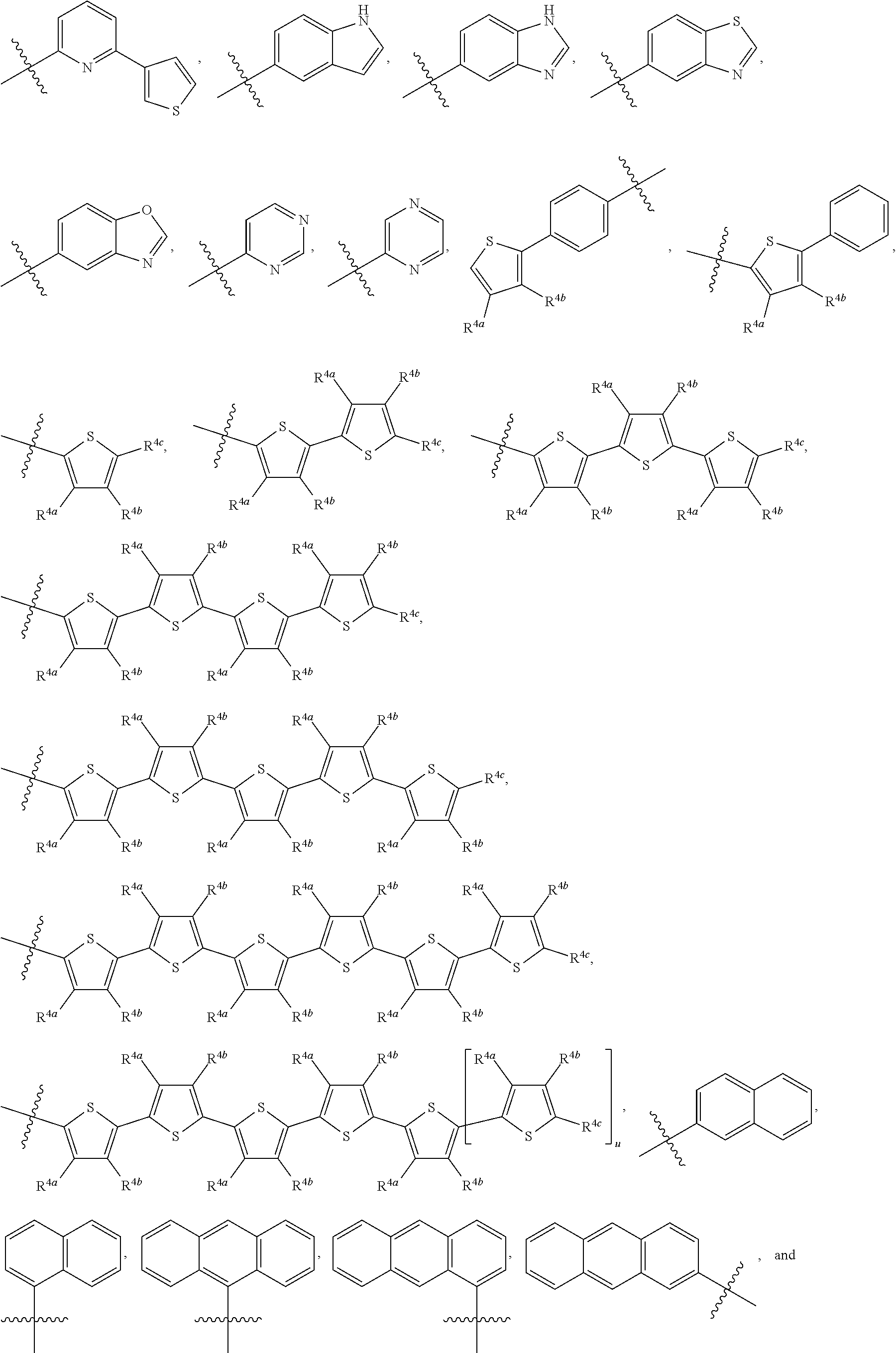

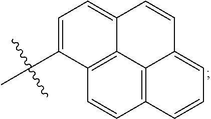 [SMILES: *c1ccc2ccc3cccc4ccc1c2c34]

u is an integer, which is preferably 1, 2, 3, 4, 5, 6, 7, 8, 9 or 10;
R^{2a}, R^{2b}, R^{2c}, R^{2d}, R^{2e}, R^{2f}, R^{2g}, R^{2h}, R²ⁱ, R^{2j}, R^{2k}, and R^{2l} at each occurrence are each independently selected from the group consisting of hydrogen, C₁₋₆ optionally substituted alkyl, C₁₋₆ optionally substituted branched alkyl, C₃₋₇ optionally substituted cycloalkyl, C₁₋₆ optionally substituted haloalkyl, C₁₋₆ optionally substituted alkoxy, CO₂R⁵, CONR⁶₂, NR⁷₂, SO₃H, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R³€, R^{3f}, R^{3g}, R^{3h} R³ⁱ, R^{3j}, R^{3k}, and R^{3l} at each occurrence are each independently selected from the group consisting of hydrogen, C₁₋₆ optionally substituted alkyl, C₁₋₆ optionally substituted branched alkyl, C₁₋₆ optionally substituted haloalkyl, C₁₋₆ optionally substituted alkoxy, optionally substituted phenyl, and CO₂R⁸;
R^{4a}, R^{4b}, and R^{4c} at each occurrence are each independently selected from the group consisting of hydrogen, C₁₋₆ optionally substituted alkyl, C₁₋₆ optionally substituted branched alkyl, C₁₋₆ optionally substituted cycloalkyl, C₁₋₆ optionally substituted haloalkyl, C₁₋₆ optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence are each independently selected from the group consisting of hydrogen and optionally substituted alkyl;
R⁶ at each occurrence are each independently selected from the group consisting of hydrogen and optionally substituted alkyl;
R⁷ at each occurrence are each independently selected from the group consisting of hydrogen and optionally substituted alkyl; and
R⁸ at each occurrence are each independently selected from the group consisting of hydrogen and optionally substituted alkyl
p is independently at each occurrence 0, 1, or 2;
q is independently at each occurrence 0, 1, or 2; and
n is 0, 1, 2, 3, 4, or 5; or
(d) formula (II):

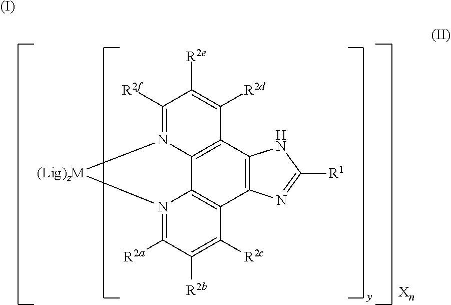

including hydrates, solvates, pharmaceutically acceptable salts, prodrugs and complexes thereof, wherein:
M is selected from the group consisting of manganese, molybdenum, rhenium, iron, ruthenium, osmium, cobalt, rhodium, iridium, nickel, platinum, and copper;
X is selected from the group consisting of Cl⁻, PF₆⁻, Br⁻, BF₄⁻, ClO₄⁻, CF₃SO₃⁻, and SO₄⁻²;
n=0, 1, 2, 3, 4, or 5;
y=1, 2, or 3;
z=0, 1, or 2;
Lig at each occurrence is independently selected from the group consisting of

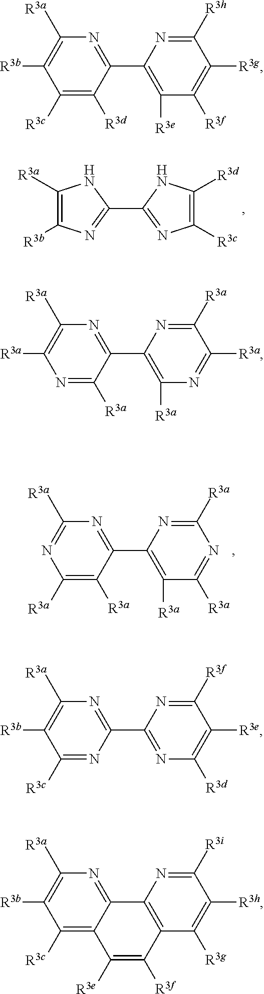

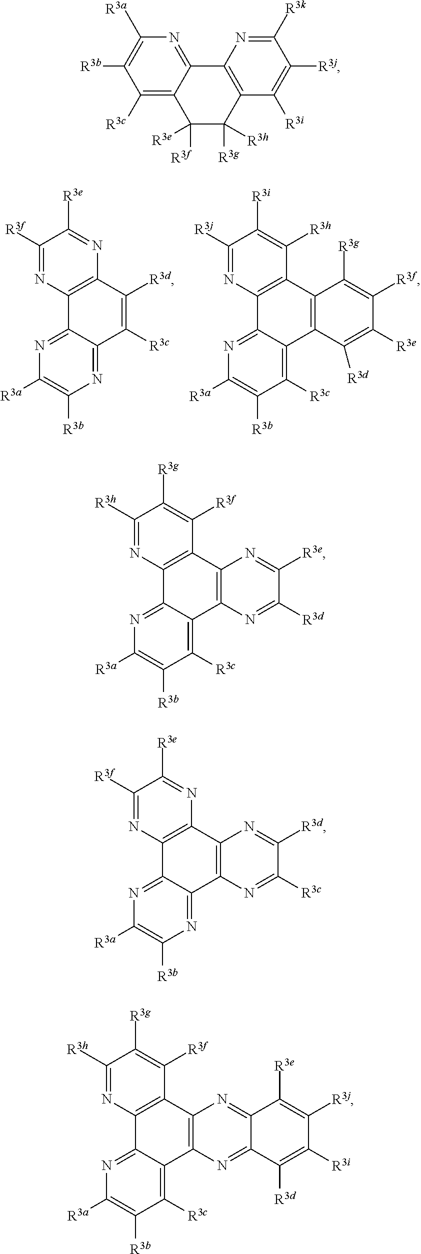

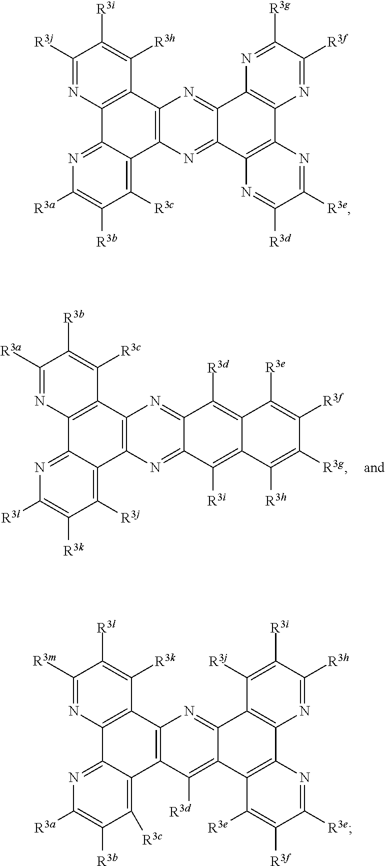

R¹ is selected from the group consisting of

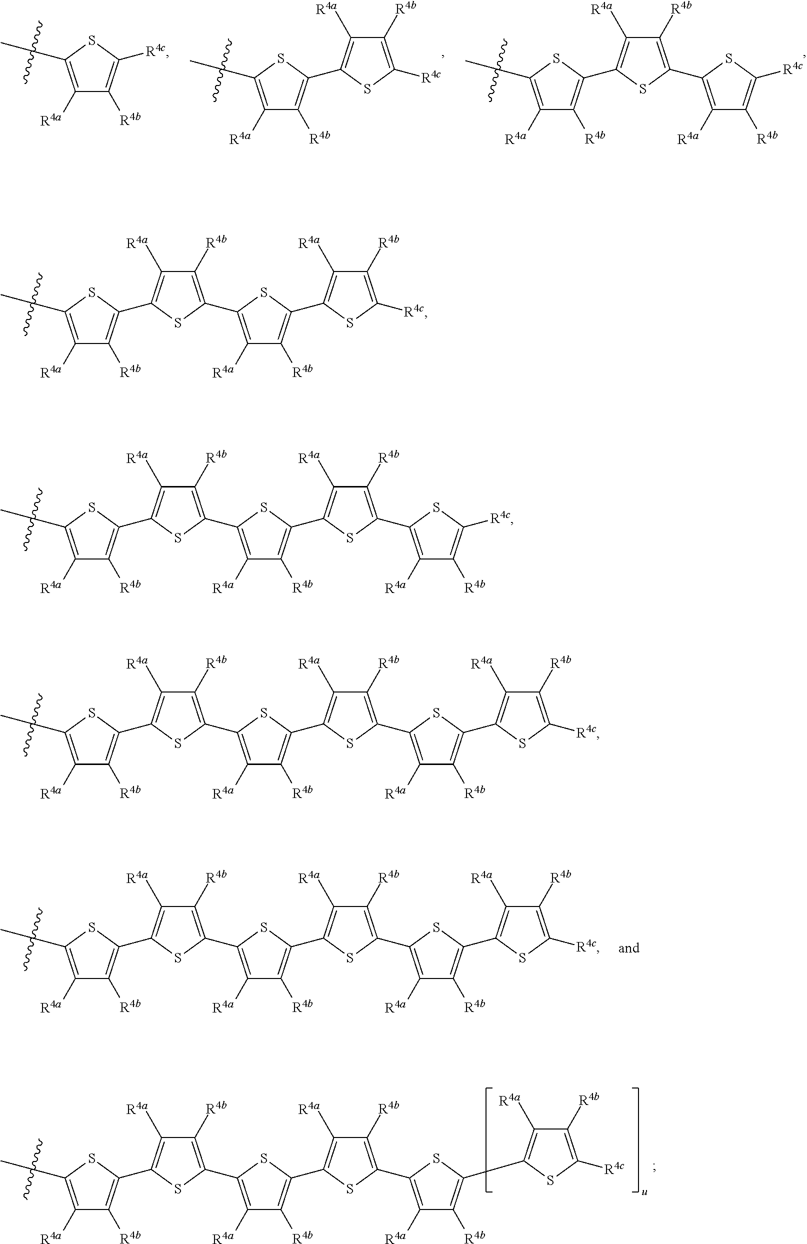

u is an integer;
R^{2a}, R^{2b}, R^{2c}, R^{2d}, R^{2e}, and R^{2f} at each occurrence are each independently selected from the group consisting of hydrogen, C₁₋₆ optionally substituted alkyl, C₁₋₆ optionally substituted branched alkyl, C₃₋₇ optionally substituted cycloalkyl, C₁₋₆ optionally substituted haloalkyl, C₁₋₆ optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h} R³ⁱ, R^{3j}, R^{3k}, R^{3l}, and R^{3m} at each occurrence are each independently selected from the group consisting of hydrogen, C₁₋₆ optionally substituted alkyl, C₁₋₆ optionally substituted branched alkyl, C₁₋₆ optionally substituted haloalkyl, C₁₋₆ optionally substituted alkoxy, and CO₂R⁸;
R^{4a}, R^{4b}, and R^{4c} at each occurrence are each independently selected from the group consisting of hydrogen, C₁₋₆ optionally substituted alkyl, C₁₋₆ optionally substituted branched alkyl, C₁₋₆ optionally substituted cycloalkyl, C₁₋₆ optionally substituted haloalkyl, C₁₋₆ optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence is independently selected from the group consisting of hydrogen and optionally substituted alkyl;
R⁶ at each occurrence is independently selected from the group consisting of hydrogen and optionally substituted alkyl;
R⁷ at each occurrence is independently selected from the group consisting of hydrogen and optionally substituted alkyl; and
R⁸ at each occurrence is independently selected from the group consisting of hydrogen and optionally substituted alkyl.

Particularly preferred PDCs include Ru(2,2′-bipyridine)₂(2-(2′,2″:5″,2″″″-terthiophene)-imidazo[4,5-f][1,10]phenanthroline); Ru(2,2′-bipyridine)₂(2-(2′,2″:5″,2″″;5″′,2″″-quaterthiophene)-imidazo[4,5-f][1,10]phenanthroline); Ru(4,4′-dimethyl-2,2′-bipyridine)₂(2-thiophenimidazo[4,5-f][1,10]phenanthroline); Ru(4,4′-dimethyl-2,2′-bipyridine)₂(2-(2′,2″-bithiophene)-imidazo[4,5-f][1,10]phenanthroline); Ru(4,4′-dimethyl-2,2′-bipyridine)₂(2-(2′,2″:5″,2′″-terthiophene)-imidazo[4,5-f][1,10]phenanthroline); Ru(4,4′-dimethyl-2,2′-bipyridine)₂(2-(2′,2″:5″,2′″;5″,2″″-quaterthiophene)-imidazo[4,5-f][1,10]phenanthroline); Ru(4,4′-di-t-butyl-2,2′-bipyridine)₂(2-thiophenimidazo[4,5-f][1,10]phenanthroline); Ru(4,4′-di-t-butyl-2,2′-bipyridine)₂(2-(2′,2″-bithiophene)-imidazo[4,5-f][1,10]phenanthroline); Ru(4,4′-di-t-butyl-2,2′-bipyridine)₂(2-(2′,2″:5″,2′″-terthiophene)-imidazo[4,5-f][1,10]phenanthroline); Ru(4,4′-dimethoxy-2,2′-bipyridine)₂(2-(2′,2″:5″,2′″-terthiophene)-imidazo[4,5-f][1,10]phenanthroline); Ru(5,5′-dimethyl-2,2′-bipyridine)₂(2-thiophenimidazo[4,5-f][1,10]phenanthroline); Ru(5,5′-dimethyl-2,2′-bipyridine)₂(2-(2′,2″-bithiophene)-imidazo[4,5-f][1,10]phenanthroline); Ru(5,5′-dimethyl-2,2′-bipyridine)₂(2-(2′,2″:5″,2′″-terthiophene)-imidazo[4,5-f][1,10]phenanthroline); Ru(6,6′-dimethyl-2,2′-bipyridine)₂(2-thiophenimidazo[4,5-f][1,10]phenanthroline); Ru(6,6′-dimethyl-2,2′-bipyridine)₂(2-(2′,2″-bithiophene)-imidazo[4,5-f][1,10]phenanthroline); Ru(6,6′-dimethyl-2,2′-bipyridine)₂(2-(2′,2″:5″,2′″-terthiophene)-imidazo[4,5-f][1,10]phenanthroline); Ru(4,4′-di(methylcarboxy)-2,2′-bipyridine)₂(2-(2′,2″:5″,2′″-terthiophene)-imidazo[4,5-f][1,10]phenanthroline); Ru(2,2′-bipyrimidine)₂(2-(2′,2″:5″,2′″-terthiophene)-imidazo[4,5-f][1,10]phenanthroline); Ru(2,2′-bipyrimidine)(4,4′-dimethyl-2,2′-bipyridine)(2-(2′,2″:5″,2′″-terthiophene)-imidazo[4,5-f][1,10]phenanthroline); Ru(1,10-phenanthroline)₂(2-thiophenimidazo[4,5-f][1,10]phenanthroline); Os(2,2′-bipyridine)₂(2-thiophenimidazo[4,5-f][1,10]phenanthroline); Os(2,2′-bipyridine)₂(2-(2′,2″-bithiophene)-imidazo[4,5-f][1,10]phenanthroline); Os(2,2′-bipyridine)₂(2-(2′,2″:5″,2′″-terthiophene)-imidazo[4,5-f][1,10]phenanthroline); Os(1,10-phenanthroline)₂(2-thiophenimidazo[4,5-f][1,10]phenanthroline); Os(1,10-phenanthroline)₂(2-(2′,2″-bithiophene)-imidazo[4,5-f][1,10]phenanthroline); Os(1,10-phenanthroline)₂(2-(2′,2″:5″,2′″-terthiophene)-imidazo[4,5-f][1,10]phenanthroline); Os(4,4′-dimethyl-2,2′-bipyridine)₂(2-thiophenimidazo[4,5-f][1,10]phenanthroline); Os(4,4′-dimethyl-2,2′-bipyridine)₂(2-(2′,2″-bithiophene)-imidazo[4,5-f][1,10]phenanthroline); Os(4,4′-dimethyl-2,2′-bipyridine)₂(2-(2′,2″:5″,2′″-terthiophene)-imidazo[4,5-f][1,10]phenanthroline); and pharmaceutically acceptable salts thereof.

### Metal-Binding Glycoproteins

The PDC is preferably administered to the patient along with a metal-binding glycoprotein. Metal-binding glycoproteins suitable for use in the invention are capable of binding transition metals and delivering to a biological target said metals and other materials complexed with said metals. The metal-binding glycoproteins are preferably capable of binding Group 8 metals and/or Group 9 metals, and most preferably Ru, Os and Rh. Most preferred are the iron-binding glycoproteins: transferrin, lactoferrin, ovotransferrin and melanotransferrin and variants thereof, with transferrin being most preferred. The glycoprotein can be purified from natural sources or can be from artificial sources. Thus, for example, the glycoprotein in certain embodiments is a recombinant transferrin, such as Apo Transferrin or OPTIFERRIN, a recombinant human transferrin available from InVitria, a division of Ventria Bioscience.

### Vaccines

Another example of contemporary immunogenic anticancer therapy is the clinical use of cancer vaccines; including, replicated viruses, such as: Oncolytic Viruses (“OVs”) and non-replicated viral vectors, which are harboring human genes, such as ADSTILADRIN (Nadofaragene Firadenovec).

OVs are an emerging class of cancer therapeutics that offer the benefits of selective replication in tumor cells, delivery of multiple eukaryotic transgene payloads, induction of immunogenic cell death and promotion of antitumor immunity and a tolerable safety profile that largely does not overlap with that of other cancer therapeutics. Ilkow et al. “From scourge to cure: tumour-selective viral pathogenesis as a new strategy against cancer.” PLOS Pathog. 10, e1003836 (2014).

OVs are thought to induce the release of Damage-Associated Molecular Patterns (“DAMPs”), eliciting a pro-inflammatory cytokine release and stimulating the activation of the innate immune system.

Concurrently, oncolytic virotherapy-induced oncolysis leads to further release of neoantigens and subsequent epitope spreading, culminating in a robust, tumor-specific adaptive immune response. Kaufman et al. “Oncolytic viruses: a new class of immunotherapy drugs.” Nat. Rev. Drug Discov. 14, 642-662 (2015).

Recombinant IFN alpha2b has pleiotropic effects that contribute to antitumor activity in Non-Muscle Invasive Bladder Cancer (“NMIBC”).

ADSTILADRIN is a non-replicating adenovirus vector harboring the human IFN alpha2b gene. When combined with the excipient Syn3, intravesical administration of the rAd-IFN results in transduction of the virus into the epithelial cell lining in the bladder. The IFN alpha2b gene is incorporated into the cellular DNA resulting in the synthesis and expression of large amounts of IFN alpha2b protein. See https://www.fda.gov/vaccines-blood-biologics/cellular-gene-therapy-products/ADSTILADRIN.

OVs can act as an in-situ cancer vaccine by inducing ICD, which leads to the spatiotemporal release of soluble tumor-associated antigens, DAMPs and pathogen-associated molecular patterns. The success of such in-situ vaccinations depends on optimal activation of the antiviral immune response; if this response is too strong, it might result in rapid viral clearance, which could limit the induction of anti-tumor immunity. See Krysko et al. “Immunogenic cell death and DAMPs in cancer therapy.” Nat. Rev. Cancer 12, 860-875 (2012), and Romo. “Cell death as part of innate immunity: cause or consequence.” Immunology 163, 399-415 (2021). Thus, achieving optimal tumor regression with an OV depends on finding the optimal balance between the antiviral immune response and the antitumor immune response.

Humoral immunity-mediated rapid neutralization of OVs, for example, by virus-specific antibodies as well as by components of the complement system, is a considerable obstacle for the activity of nearly all OVs. This challenge might be especially relevant for OVs derived from endemic viruses, such as HSVs or adenoviruses, because patients previously exposed to viruses from the same family might have pre-existing cross-reactive antibodies that can impair effective viral replication. See Wakimoto et al. “The complement response against an oncolytic virus is species-specific in its activation pathways.” Mol. Ther. 5, 275-282 (2002), and Ikeda et al. “Complement depletion facilitates the infection of multiple brain tumors by an intravascular, replication-conditional herpes simplex virus mutant.” J. Virol. 74, 4765-4775 (2000). Furthermore, patients with advanced-stage cancers are likely to require multiple OV injections and the emergence of neutralizing antibodies might preclude this.

The use of cancer vaccines in general and oncolytic virotherapy in particular will benefit when combined with oxidative stress mediated therapy provided by PDCs of the invention, which will enable a strong maintenance of the balance between antiviral and antitumor immunity.

It should be noted that ADSTILADRIN-induced amplification of interferon signalling, once the targeted JAK/STAT pathway (which regulates not only proteins involved in inhibition of cancer cells proliferation, but also apoptosis) is activated, can lead to compensatory negative signalling resulting an abrupt interferon downregulation to maintain homeostasis and normal cell activities. This reverse mechanism will compromise the efficacy of ADSTILADRIN. For example, Suppressor Of Cytokine Signalling 1 (“SOCS1”) can decrease the phosphorylation level of JAK1 and STAT1. Ubiquitin Specific Peptidase 18 (“USP18”) interferes with IFN-I signalling by inducing degradation of ISG15 and disrupting IFNAR2-JAK1 binding. See Luan et al. “miR-150-based RNA interference attenuates tubulointerstitial fibrosis through the SOCS1/JAK/STAT pathway In Vivo and In Vitro.” Mol Ther Nucleic Acids. 2020; 22:871-84. Combined therapy, including the administration of at least one PDC of the invention in combination with ADSTILADRIN will addressing the drug (ADSTILADRIN/IFN-α2b) resistance and nonspecific inflammatory responses caused by IFN-α2b. For example, via an alternative to IFN-α2b, synergistic stimulation of JAK/STAT pathway by PDC-based therapy, which could be achieved via upregulation of calreticulin (O'Sullivan et al. “JAK-STAT signaling in the therapeutic landscape of myeloproliferative neoplasms.” Mol Cell Endocrinol; 2017 Aug. 15; 451:71-79) by the oxidative stress induced by PDC activation.

It is also a valid assumption that T cells elicit a particularly robust response if they recognize so-called mutated Tumor-Specific Antigens (“mTSAs”). These antigens arise from somatic mutations (such as single-nucleotide variants, small insertions and deletions, or gene fusions) specifically in malignant cells, thus giving rise to MHC class I (MHC I)-binding peptides that are exclusively found on cancer cells but not on non-malignant cells. Ahmadzadeh et al. “Tumor antigen-specific CD8 T cells infiltrating the tumor express high levels of PD-1 and are functionally impaired.” Blood. 2009; 114:1537-1544; therefore, as the PDC is spatially localized in the cytoplasm of cancer cells (see FIG. 9), upon the PDC activation, oxidatively damaged tumor cells undergo cell-death pathways and release DAMPs and TSAs; hence, likely synergized efficacy of ICIs.

Moreover, inflammation caused by stress-related events induced by the PDC, can lead to the expression of non-mutated Tumor Antigens (“nmTAs”). In addition to mTSAs, the expression of nmTAs can play an important synergistic role in immunosurveillance (see Table 3 below). Indeed, experimental, and epidemiological evidence suggests that stress-related nmTAs play a major role in the natural and therapy-modulated evolution of malignant disease. See Xiao et al. “Activator protein 1 suppresses antitumor T-cell function via the induction of programmed death 1.” Proc Natl Acad Sci USA. 2012; 109:15419-15424, and Schumacher et al. “Cancer neoantigens.” Annual review of immunology 37 (2019): 173-200.

Table 3. Proposed Mechanisms of Anticancer Effect of PDCs in Combination with One of the PD-L1/PD-L2 Checkpoint Inhibitors (Superior or Maximized Anti-Tumor Immunity).

| | | | | | |
|---|---|---|---|---|---|
| Malignant cell | PDC-induced | DAMP, TAAs + | Innate and | Attract APCs | Immune response |
| | immunogenic | TSAs. | adaptive | (such as DCs and | against |
| | (oxidative) | “Sterile” | checkpoint | Mφ, precursors. | immunodominant |
| | stress/damage/death | inflammatory | downregulation | Mature and | epitopes. |
| | of malignant | responses | with upregulation | active Aps. | Broad immune |
| | cells (ICD) | | of chemokines, | B-cells activation | response against |
| | | | such as, CXCL10 | Facilitation of | tumor specific |
| | | | | cross- | antigens. |
| | | | | presentation. | Long-lasting |
| | | | | T-cells | “amplifying” |
| | | | | recognition of | anticancer |
| | | | | multiple TAAs + | immunity |
| | | | | TSAs epitopes | |
| | | | | and enhanced T- | |
| | | | | cells priming to | |
| | | | | tumor-specific | |
| | | | | CD8+ cells | |

First, the proposed combination of anticancer therapies should be capable of eliciting suitable stress (and damage) responses in cancer cells that ideally comprise a combination of danger signals (DAMPs) that are able to alert innate immune effectors, in particular Dendritic Cells (“DCs”) and macrophages that result in the expression of neoantigens such as stress-associated self-mTAs and nmTAs antigens. Second, cytotoxic T lymphocytes educated by antigen presenting cells, such as macrophages and/or DCs would initiate a synergistic or additive immune response to ICIs and/or OVs that can lead to facilitating an advanced immune response against cancer cells, thus minimizing the resistance and/or loss of anticancer activity of ICIs and/or OVs. Third, the resulting immune response would eliminate all malignant (mutated and/or unmutated) tumor cells, including cancer stem cells.

PD-1 is an inhibitor of both adaptive and innate immune responses and is expressed on activated T, Natural Killer (“NK”) and B lymphocytes, macrophages, Dendritic Cells (“DCs”) and monocytes. (See Ahmadzadeh et al)

Of note, PD-1 is highly expressed on tumor-specific T cells. (See Ahmadzadeh et al)

Also, it has been reported that cancer cell leakage increases the expression of the c-FOS subunit of AP1; thereby, increasing the expression of PD-1 and, hence, could contribute to suppression of immunosurveillance and indigenous anti-cancer responses. (See Xiao et al)

A combination of oxidative stress therapy with a PDC of the invention and monoclonal antibodies, such as Cetrelimab and/or Sasanlimab, which are directed against the negative immunoregulatory human cell surface receptor programmed cell death 1 protein (PD-1, PCDC-1), with potential immune checkpoint inhibitory and antineoplastic activity is desirable. Our research results suggest that PDC treated cancer cells undergo ICD, suppression of CD47 signaling and activation of CD8+ T cells. These anticancer effects induced by the inventive PDCs will be synergized upon administration of immunotherapeutic agents, such as MABs and/or targeting ICIs therapeutics, for treatment of mammalian tumors.

The invention will be illustrated in more detail with reference to the following examples, but it should be understood that the present invention is not deemed to be limited thereto.

### EXAMPLES

MYC oncogene, deregulated in >50% of human cancers, has been found to upregulate both CD47 and PD-L1 expression on the surface of tumor cells by binding to their promoters. See Yang et al. “PD-L1 and CD47 co-expression predicts survival and enlightens future dual-targeting immunotherapy in non-small cell lung cancer.” Thorac Cancer. 2021 June; 12 (11): 1743-1751.

CD47, a cell surface molecule in the immunoglobulin superfamily, is another established target overexpressed on various malignant cells and has been described colloquially as the regulator of a “Don't-Eat-Me” signal. See Yang et al. and Wang et al. “Blockade of dual immune checkpoint inhibitory signals with a CD47/PD-L1 bispecific antibody for cancer treatment.” Theranostics. 2023; 13 (1): 148-160. CD47 binds to several proteins; specifically: Signal Regulatory Protein alpha (“SIRPα”), Signal Regulatory Protein gamma (“SIRPγ”), integrins and thrombospondin-1; hence, when CD47 is bound to its receptor, SIRPα (present on various myeloid cells in the microenvironment of cancerous tumors), CD47 acts as an innate inhibitory checkpoint by interrupting the phagocytosis of tumor cells and downstream activation of innate responses.

By suppressing innate immune activation, presentation of tumor antigens and priming of T cell responses, CD47 enables tumor cells to escape both innate and adaptive immune surveillance.

If CD47 could be downregulated, then ICIs would be more effective in the destruction of cancer cells.

CD47 overexpression has been shown to act as an inhibitor to PD-1/PD-L1 therapy effectiveness in preclinical models (see below), thus, CD47 has become an attractive target, with various approaches to block or downregulate CD47/SIRPα interactions in clinical development. (See Wang et al)

Testing was therefore conducted to evaluate the autoimmune properties of an exemplary PDC of the invention, Ru(4,4′-dimethyl-2,2′-bipyridine)₂(2-(2′,2″:5″,2′-terthiophene)-imidazo[4,5-f][1,10]phenanthroline) chloride (i.e.: TLD-1433) with co-administered apo-transferrin (RUTHERRIN, available from Theralase Technologies, Inc., Toronto, Canada) and without transferrin (RUVIDAR, Theralase Technologies, Inc.).

### Example 1

Human glioma (U87) or human bladder cancer (T24) cells were treated with RUTHERRIN for 4 hours, then treated with green (530 nm) light PDT (90 J/cm²). 1 hour after treatment, cells were analyzed for CD47 expression by flow cytometry.

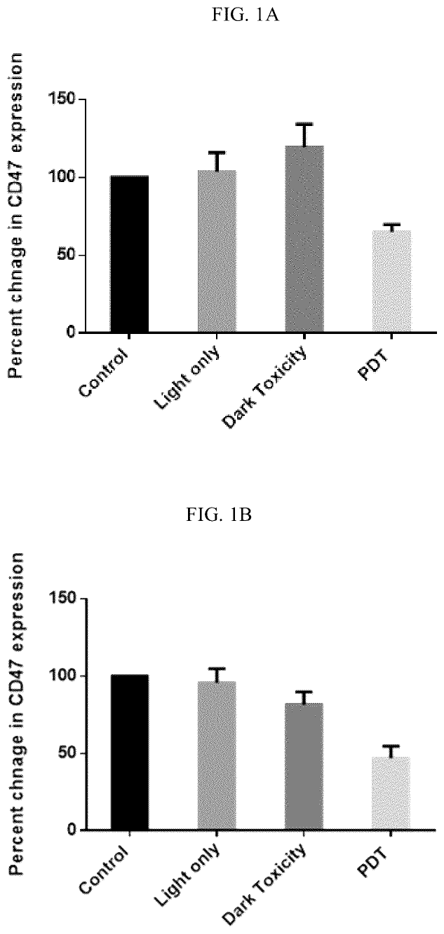

RUTHERRIN downregulated CD47 in U87 human glioma cells (FIG. 1A) and in T24 human urinary bladder cancer cells (FIG. 1B).

### Example 2

Rat Glioma (“RG2”) cells were treated with RUTHERRIN for 4 hours, then treated with green (530 nm) light PDT (90 J/cm²). One (1) hour after treatment, cells were analyzed for cell surface Calreticulin (“CRT”) expression by flow cytometry. Control included untreated cells, drug only treated cells and light only treated cells, which showed similar CRT expression and were pooled together into one (1) control bar.

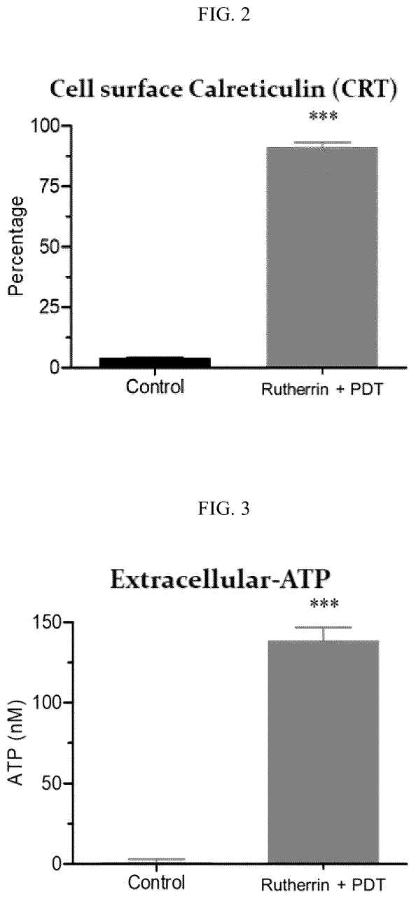

RUTHERRIN upregulated calreticulin (FIG. 2).

### Example 3

RG2 cells were treated with RUTHERRIN for 4 hours, then treated with green (530 nm) light PDT (90 J/cm²). One (1) hour after treatment, cell supernatant was collected and used to quantify ATP release. Control included untreated cells, drug only treated cells, and light only treated cells, which showed low ATP production and were pooled together into one (1) control bar.

RUTHERRIN upregulated ATP (FIG. 3).

### Example 4

RG2 cells were treated with RUTHERRIN for 4 hours, then treated with green (530 nm) light PDT (90 J/cm²). One (1) hour after treatment, cells were collected for RNA isolation and analysis of gene expression using RT-PCR. Gene expression was normalized to untreated controls expressed as 1 to show fold change in expression with RUTHERRIN+PDT.

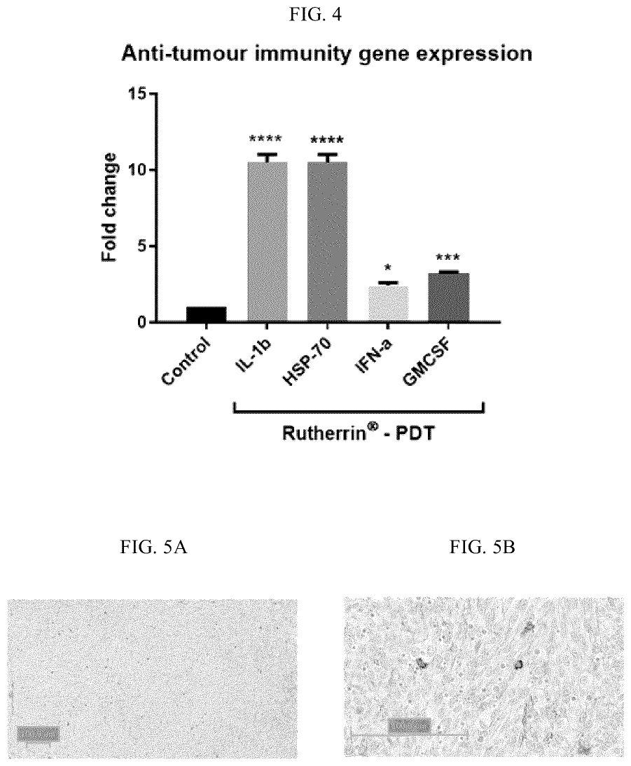

Anti-tumor immunity (IL-1b, HSP-70, INF-a and GMCSF gene expression), which is involved in priming CD4⁺ and CD8⁺ T cells RUTHERRIN (FIG. 4).

### Example 5

RG2 cells were implanted surgically into rats. Following confirmation of brain tumour establishment by MRI, rats were injected intravenously or not with RUTHERRIN (10 mg/kg). 4 hours later, brain tumors were treated with NIR (808 nm) light PDT (600 J/cm²). Brain tissues were collected and analyzed by immunohistochemistry for CD8⁺ T cells quantification in brain sections.

RG2 cells were treated ex vivo with RUTHERRIN and PDT (green light, 530 nm, 90 J/cm²) to prepare a cell vaccine. Treated cells (>99% dead) were injected subcutaneously twice at −8 and −5 days before live RG2 cell implantation in rat brains on day one (1). Control was a second group of rats injected subcutaneously with PBS instead. Following tumor establishment, brain tissues were collected and analyzed by immunohistochemistry for CD8⁺ T cells quantification in brain sections.

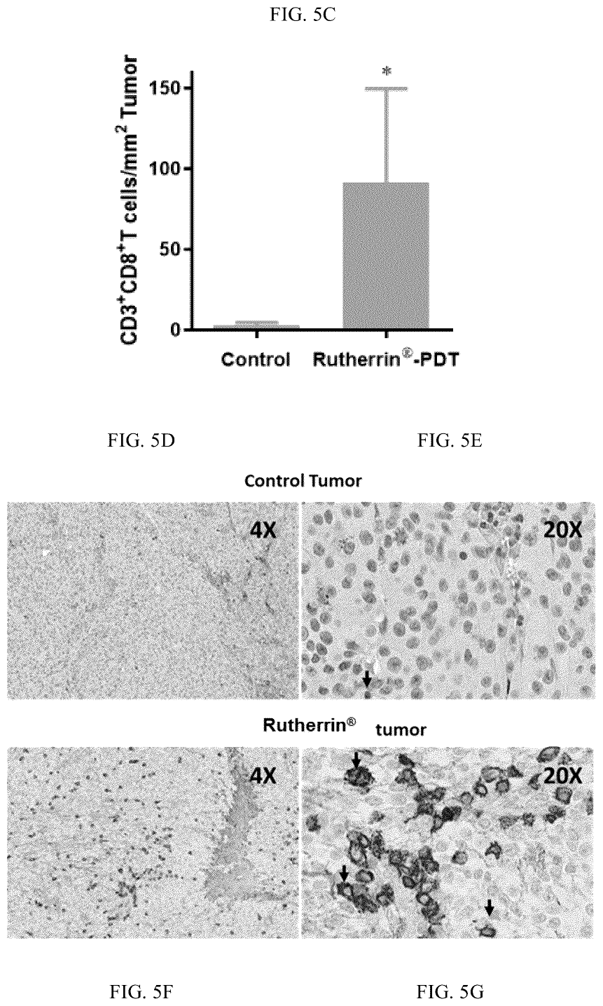

RUTHERRIN therapy activated CD8+ cells in tumor immune microenvironment (in glioblastoma). See FIGS. 5A, 5B, 5C, 5D, 5E, 5F and 5G.

### Example 6

A549 human Non-Small Cell Lung Cancer (“NSCLC”) cells were treated with RUVIDAR or RUTHERRIN for four (4) hours, then cells were treated with radiation or left untreated and then incubated further overnight before cell collection and analysis of CD47 expression by flow cytometry. Both RUVIDAR or RUTHERRIN downregulated CD47 expression without radiation treatment, and also significantly reduced expression when radiation treatment was added. Moreover, RUTHERRIN showed a trend reduction in expression more than RUVIDAR, although not statistically significant.

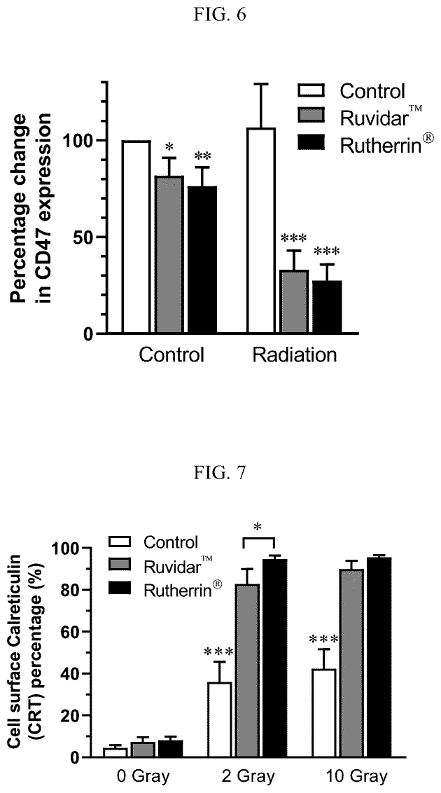

NSCLC cells treated with RUVIDAR or RUTHERRIN-CD47 expression (FIG. 6)

### Example 7

A549 human NSCLC cells were treated with RUVIDAR or RUTHERRIN for four (4) hours, then cells were treated with radiation or left untreated and then incubated further for one (1) hour before cell collection and analysis of cell surface by flow cytometry. Radiation alone induced a mild increase in CRT expression, while radiation combined with RUVIDAR or RUTHERRIN significantly increased CRT surface expression above 90%, and RUTHERRIN showed higher expression of surface CRT than RUVIDAR, which was only significant with 2 Grays of radiation.

NSCLC cells treated with RUVIDAR or RUTHERRIN-Calreticulin (CRT) expression (FIG. 7)

### Example 8

A549 human NSCLC cells were treated with RUVIDAR or RUTHERRIN for four (4) hours, then cells were treated with radiation or left untreated, and then incubated further for one (1) hour before collection of supernatant for ATP secretion quantification. Radiation alone did not cause any ATP release, while radiation combined with RUVIDAR or RUTHERRIN significantly increased ATP release. RUTHERRIN combined with radiation showed a significantly higher ATP release than RUVIDAR+radiation.

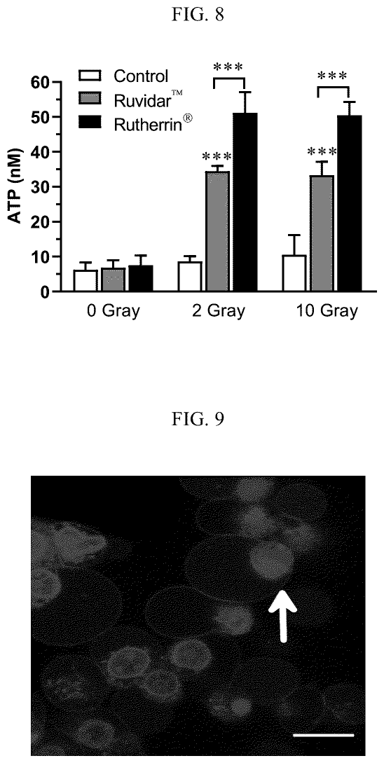

NSCLC cells treated with RUVIDAR or RUTHERRIN-ATP secretion quantification (FIG. 8)

Based on Examples 1 to 5, combination therapy with: (a) a PDC of the invention, which has a dual checkpoint downregulating effect on CD47 and Cytotoxic T-Lymphocyte-Associated protein 4 (“CTLA-4”) checkpoints; and (b) at least one of the selective PD-L1 and/or PDL-2 checkpoint inhibitors should maximize antitumor immunity and optimize the long-lasting anticancer therapeutic effect. The combination therapy should therefore exhibit superior anti-tumor results compared with a single-agent therapy.

Moreover, the lack of PD-L1 or PD-L2 ICIs efficacy in some patients could be attributed to a paucity of pre-existing immune reactive cells within the tumor immune microenvironment (Example 5), which limits the beneficial effects of ICI. In these circumstances, causing oxidative stress/damage by treatment with a PDC of the invention to attract lymphocytes before implementation of ICI is expected to enhance outcomes.

While the invention has been described in detail and with reference to specific examples thereof, it will be apparent to one skilled in the art that various changes and modifications can be made therein without departing from the spirit and scope thereof.

What is claimed is:

1. A method for treating a condition associated with hyperproliferating cells, said method comprising the steps of:
(a) administering to a patient having the condition an immunotherapeutic agent in an anti-tumor effective amount;
(b) administering to the patient a photodynamic compound; and
(c) administering radiation to the patient in whom the immunotherapeutic agent and the photodynamic compound have been administered to treat the condition.

2. The method of claim 1, wherein the condition is cancer.

3. The method of claim 2, wherein the immunotherapeutic agent is an anti-PD-1 monoclonal antibody, an anti-PD-L1 monoclonal antibody or an anti-PD-L2 monoclonal antibody.

4. The method of claim 2, wherein the immunotherapeutic agent is a cancer vaccine.

5. The method of claim 2, wherein the immunotherapeutic agent is at least one member selected from the group consisting of: idecabtagene vicleucel, lisocabtagene maraleucel, tisagenlecleucel, brexucabtagene autoleucel, axicabtagene ciloleucel, ciltacabtagene autoleucel, atezolizumab, avelumab, dostarlizumab, durvalumab, ipilimumab, nivolumab, pembrolizumab, pucotenlimab, pidilizumab, cemiplimab, spartalizumab, camrelizumab, cetrelimab, toripalimab, sintilimab, sasanlimab, dostarlizumab, SHR-1210, PDR001, MGA012, PDR001, AB122, AMP-224, JTX-4014, BGB-108, BCD-100, BAT1306, LZM009, AK105, HLX10, bevacizumab, trastuzumab, onartuzumab, erdafitinib, enapotamab, daratumumab, FAZ053, KN035, CS1001, SHR-1316, CBT-502, A167, STI-A101, CK-301, BGB-A333, MSB-2311, HLX20, LY3300054, sipuleucel-T, Bacillus Calmette-Guérin vaccine, nadofaragene firadonevec and T-VEC.

6. The method of claim 2, wherein the photodynamic compound is represented by one of the following formulas:
(i) formula (I): 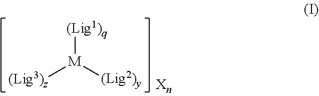
including hydrates, solvates, pharmaceutically acceptable salts, prodrugs and complexes thereof, wherein:
M at each occurrence is independently selected from the group consisting of osmium, ruthenium and rhodium;
X is selected from the group consisting of Cl⁻, PF₆⁻, Br⁻, BF₄⁻, ClO₄⁻, CF₃SO₃⁻, and SO₄⁻²;
n=0, 1, 2, 3, 4, or 5;
q is independently at each occurrence 0, 1, or 2;
y is independently at each occurrence 0, 1, or 2;
z is independently at each occurrence 1, 2, or 3;
Lig¹ is a bidentate ligand that at each occurrence is each independently selected from the group consisting of 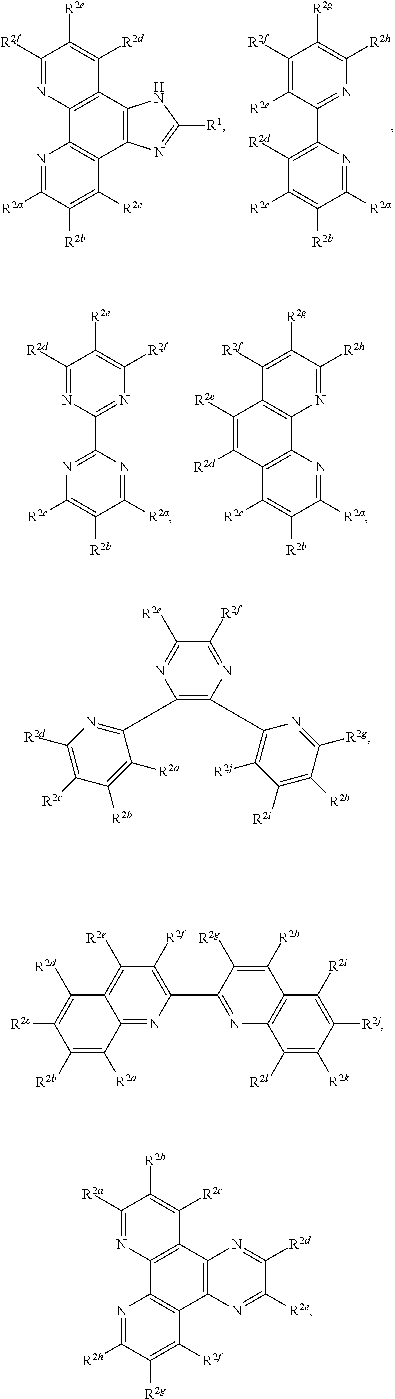 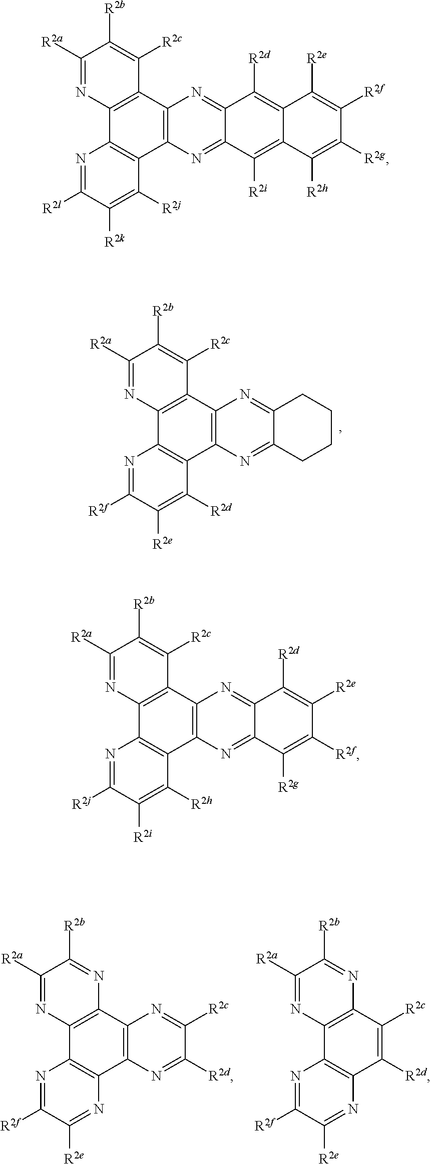 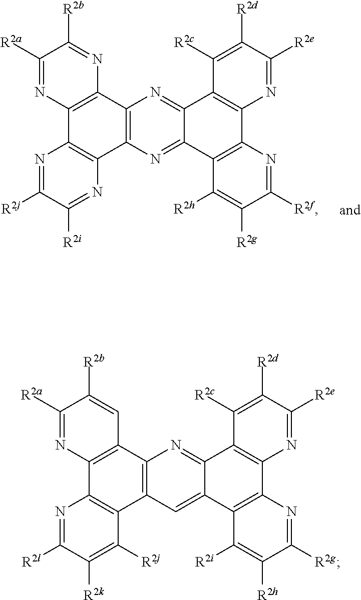
Lig² is a bidentate ligand that at each occurrence is each independently selected from the group consisting of 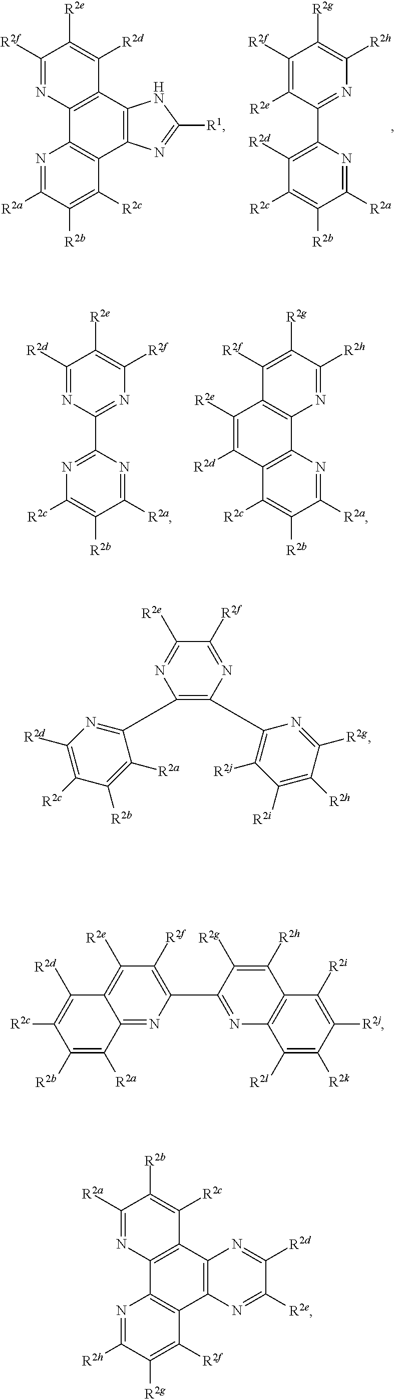 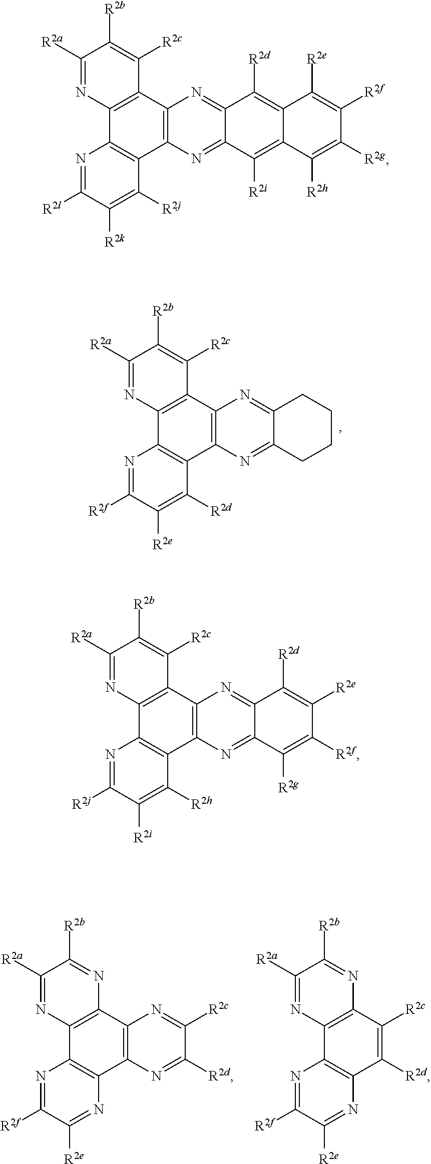 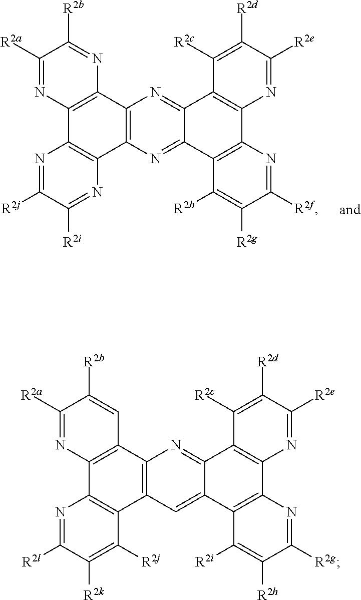
Lig³ is a bidentate ligand that at each occurrence is each independently selected from the group consisting of 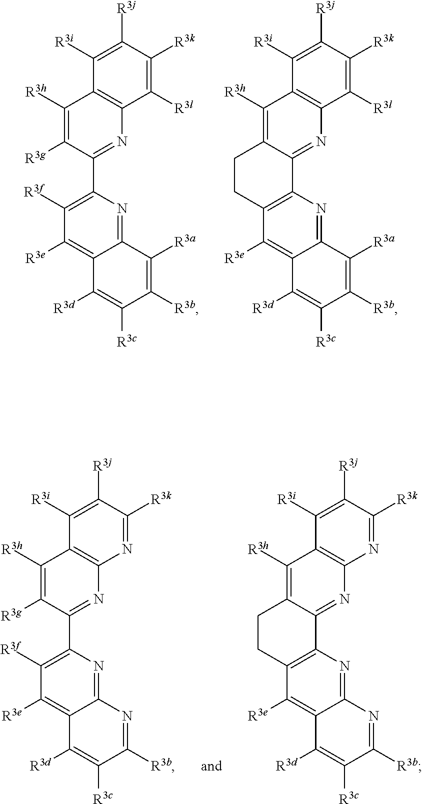
R¹ is selected from the group consisting of hydrogen, optionally substituted phenyl, optionally substituted aryl, optionally substituted heteroaryl, 4-pyridyl, 3-pyridyl, 2-thiazole, 2-pyrolyl, 2-furanyl, 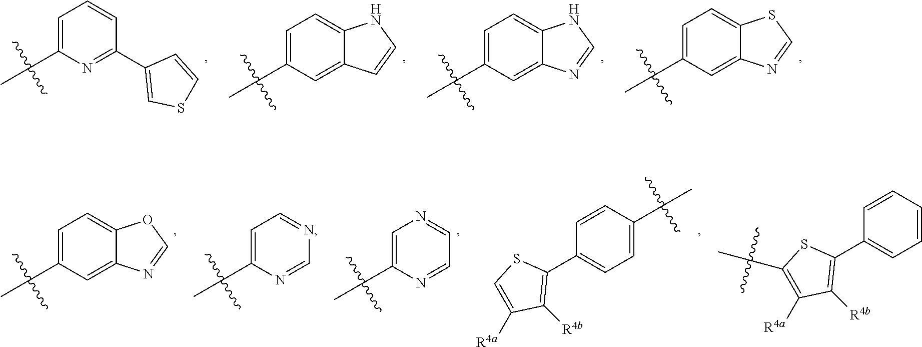 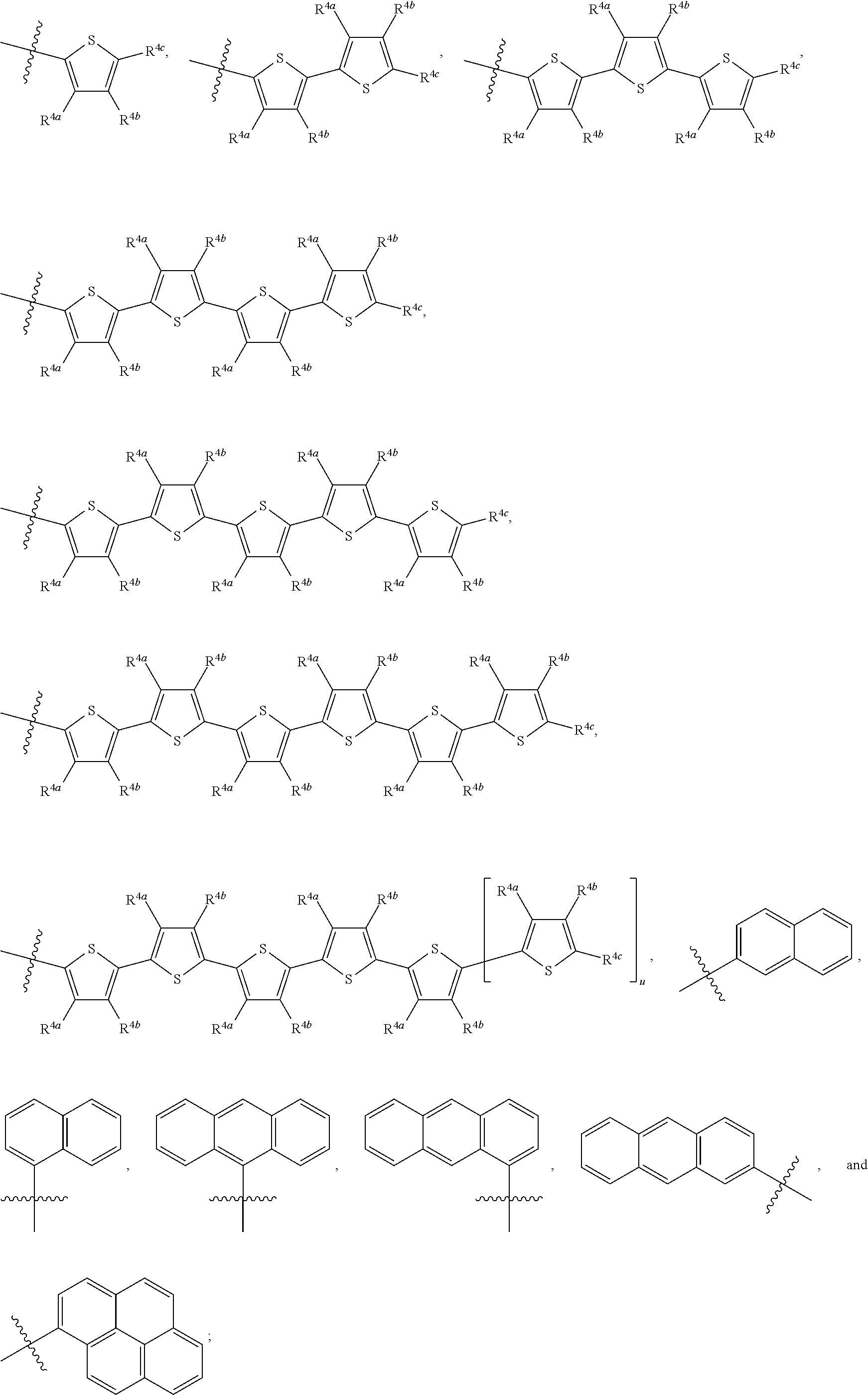
u is an integer;
R^{2a}, R^{2b}, R^{2c}, R^{2d}, R^{2e}, R^{2f}, R^{2g}, R^{2h}, R²ⁱ, R^{2j}, R^{2k}, and R^{2l} at each occurrence are each independently selected from the group consisting of hydrogen, C₁₋₆ optionally substituted alkyl, C₁₋₆ optionally substituted branched alkyl, C₃₋₇ optionally substituted cycloalkyl, C₁₋₆ optionally substituted haloalkyl, C₁₋₆ optionally substituted alkoxy, CO₂R⁵, CONR⁶₂, NR⁷₂, SO₃H, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h} R³ⁱ, R^{3j}, R^{3k}, and R^{3l} at each occurrence are each independently selected from the group consisting of hydrogen, C₁₋₆ optionally substituted alkyl, C₁₋₆ optionally substituted branched alkyl, C₁₋₆ optionally substituted haloalkyl, C₁₋₆ optionally substituted alkoxy, optionally substituted phenyl, and CO₂R⁸;
R^{4a}, R^{4b}, and R^{4c} at each occurrence are each independently selected from the group consisting of hydrogen, C₁₋₆ optionally substituted alkyl, C₁₋₆ optionally substituted branched alkyl, C₁₋₆ optionally substituted cycloalkyl, C₁₋₆ optionally substituted haloalkyl, C₁₋₆ optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence are each independently selected from the group consisting of hydrogen and optionally substituted alkyl;
R⁶ at each occurrence are each independently selected from the group consisting of hydrogen and optionally substituted alkyl;
R⁷ at each occurrence are each independently selected from the group consisting of hydrogen and optionally substituted alkyl; and
R⁸ at each occurrence are each independently selected from the group consisting of hydrogen and optionally substituted alkyl.
(ii) formula (VI): 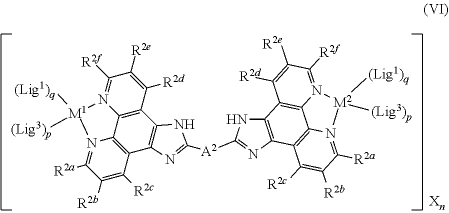
including hydrates, solvates, pharmaceutically acceptable salts, prodrugs and complexes thereof wherein;
M¹ and M² at each occurrence is independently selected from the group consisting of osmium, manganese, molybdenum, rhenium, ruthenium, iron, cobalt, rhodium, iridium, nickel, platinum, and copper;
A² is selected from the group consisting of 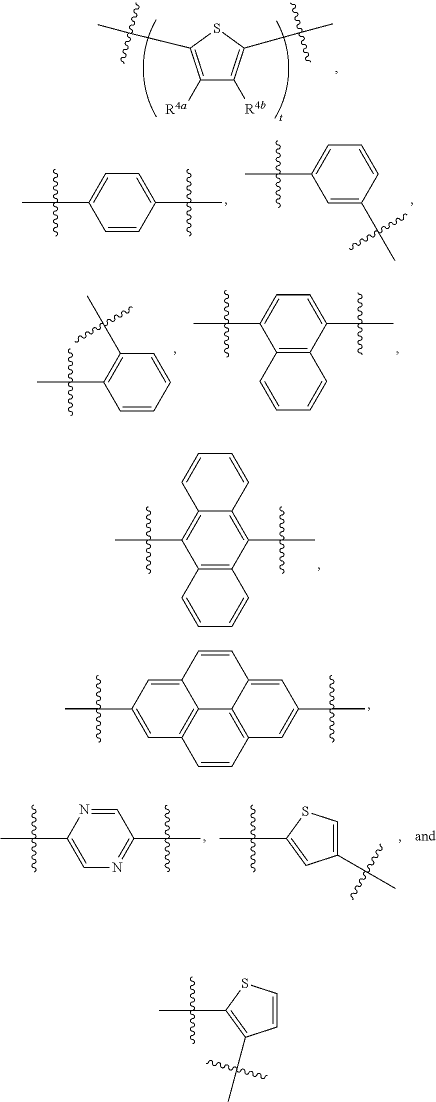
t is an integer;
(iii) formula (VIIa): 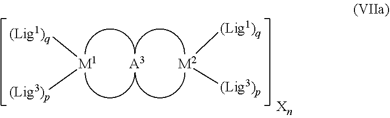
including hydrates, solvates, pharmaceutically acceptable salts, prodrugs and complexes thereof wherein:
A³ is selected from the group consisting of 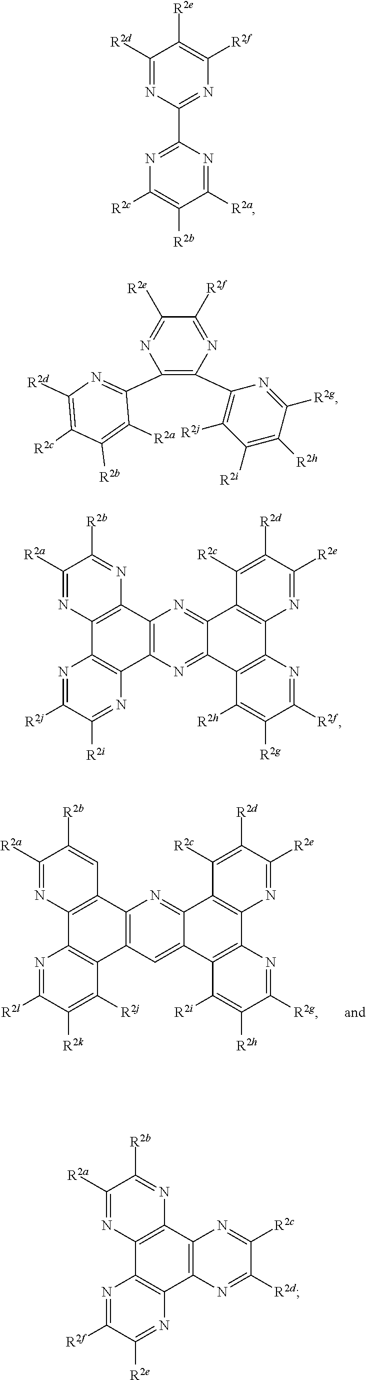
Lig¹ is a bidentate ligand that at each occurrence is each independently selected from the group consisting of 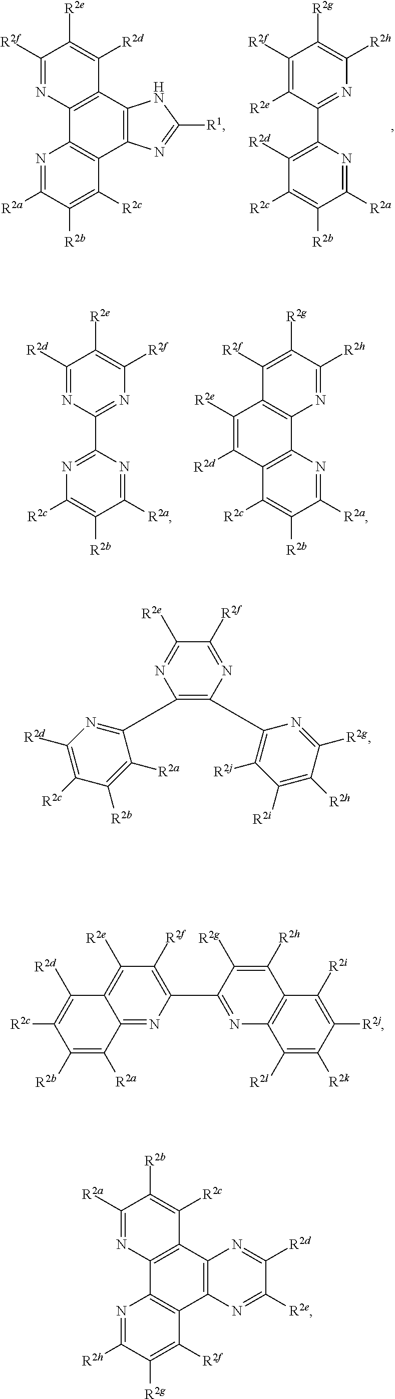 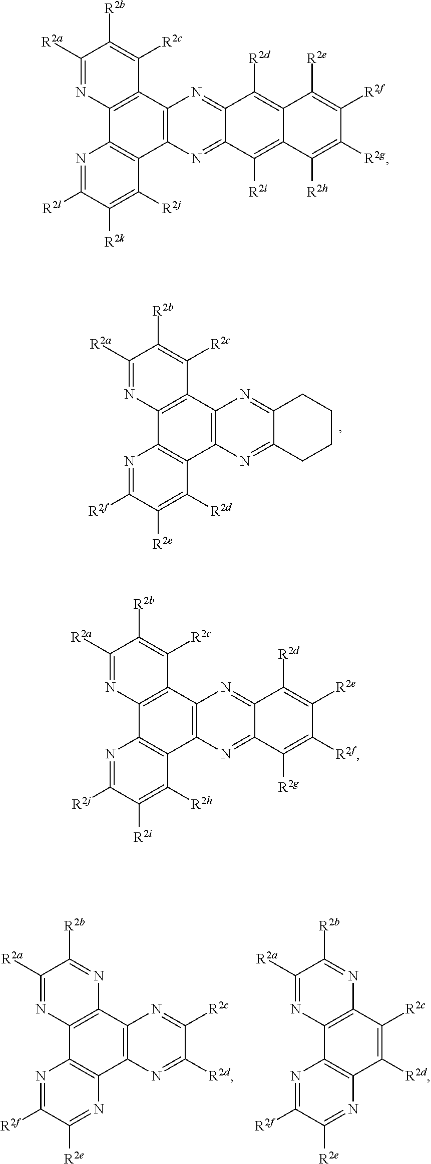 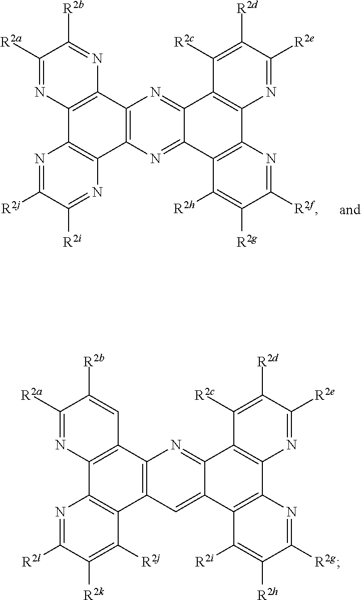
Lig³ is a bidentate ligand that at each occurrence is each independently selected from the group consisting of 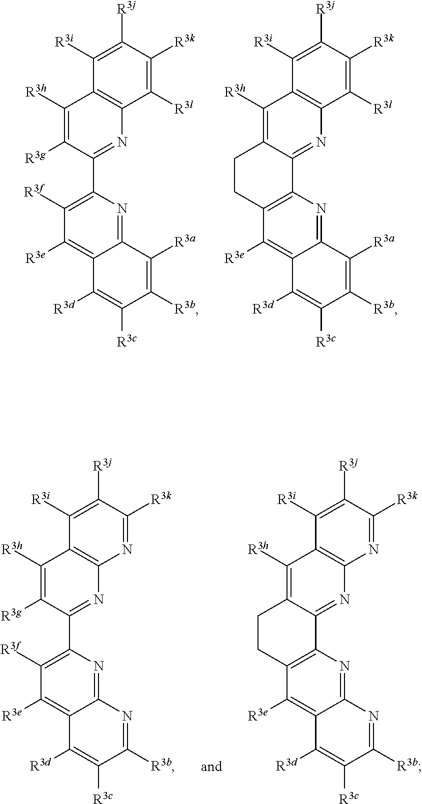
R¹ is selected from the group consisting of hydrogen, optionally substituted phenyl, optionally substituted aryl, optionally substituted heteroaryl, 4-pyridyl, 3-pyridyl, 2-thiazole, 2-pyrolyl, 2-furanyl, 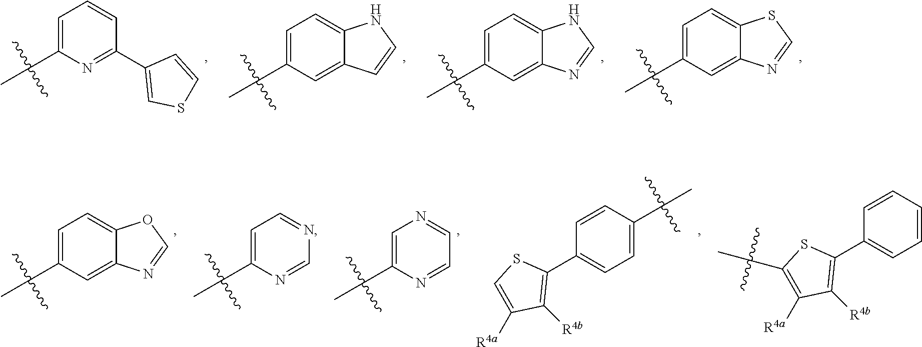 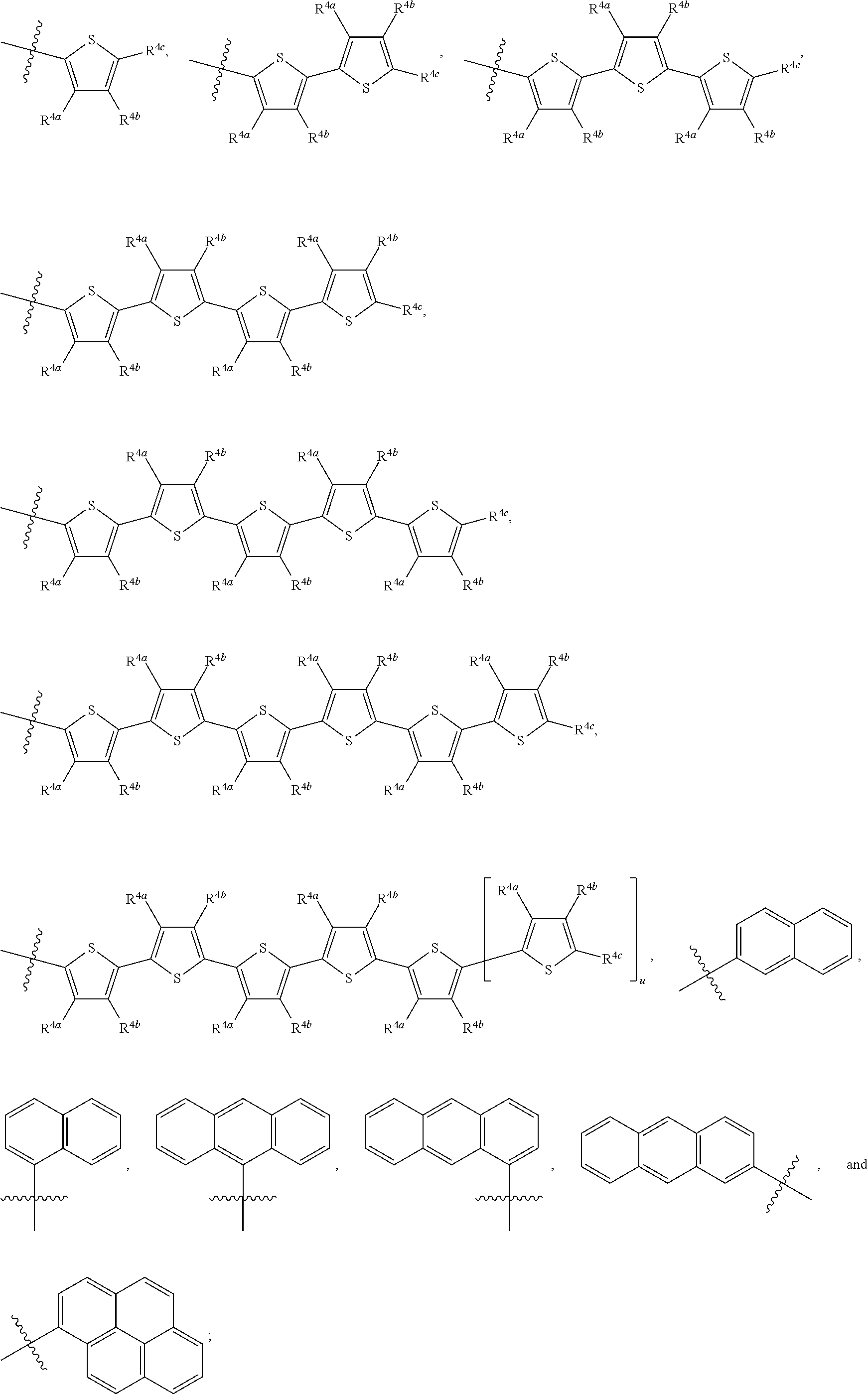
u is an integer;
R^{2a}, R^{2b}, R^{2c}, R^{2d}, R^{2e}, R^{2f}, R^{2g}, R^{2h}, R²ⁱ, R^{2j}, R^{2k}, and R^{2l} at each occurrence are each independently selected from the group consisting of hydrogen, C₁₋₆ optionally substituted alkyl, C₁₋₆ optionally substituted branched alkyl, C₃₋₇ optionally substituted cycloalkyl, C₁₋₆ optionally substituted haloalkyl, C₁₋₆ optionally substituted alkoxy, CO₂R⁵, CONR⁶₂, NR⁷₂, SO₃H, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h} R³ⁱ, R^{3j}, R^{3k}, and R^{3l} at each occurrence are each independently selected from the group consisting of hydrogen, C₁₋₆ optionally substituted alkyl, C₁₋₆ optionally substituted branched alkyl, C₁₋₆ optionally substituted haloalkyl, C₁₋₆ optionally substituted alkoxy, optionally substituted phenyl, and CO₂R⁸;
R^{4a}, R^{4b}, and R^{4c} at each occurrence are each independently selected from the group consisting of hydrogen, C₁₋₆ optionally substituted alkyl, C₁₋₆ optionally substituted branched alkyl, C₁₋₆ optionally substituted cycloalkyl, C₁₋₆ optionally substituted haloalkyl, C₁₋₆ optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence are each independently selected from the group consisting of hydrogen and optionally substituted alkyl;
R⁶ at each occurrence are each independently selected from the group consisting of hydrogen and optionally substituted alkyl;
R⁷ at each occurrence are each independently selected from the group consisting of hydrogen and optionally substituted alkyl; and
R⁸ at each occurrence are each independently selected from the group consisting of hydrogen and optionally substituted alkyl
p is independently at each occurrence 0, 1, or 2;
q is independently at each occurrence 0, 1, or 2; and
n is 0, 1, 2, 3, 4, or 5.
(iv) formula (II): 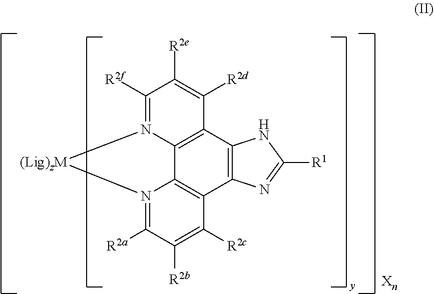
including hydrates, solvates, pharmaceutically acceptable salts, prodrugs and complexes thereof, wherein:
M is selected from the group consisting of manganese, molybdenum, rhenium, iron, ruthenium, osmium, cobalt, rhodium, iridium, nickel, platinum, and copper;
X is selected from the group consisting of Cl⁻, PF₆⁻, Br⁻, BF₄⁻, ClO₄⁻, CF₃SO₃⁻, and SO₄⁻²;
n=0, 1, 2, 3, 4, or 5;
y=1, 2, or 3;
z=0, 1, or 2;
Lig at each occurrence is independently selected from the group consisting of 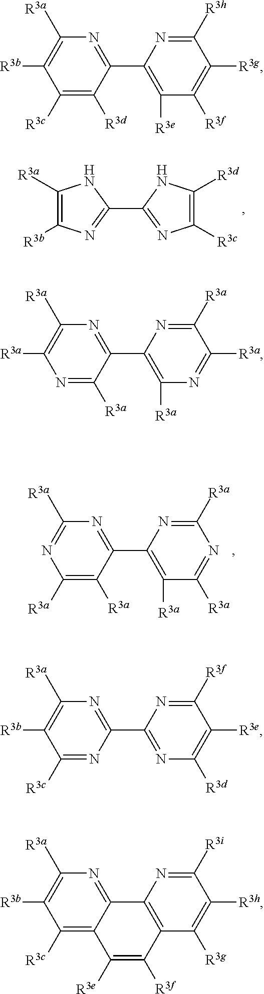 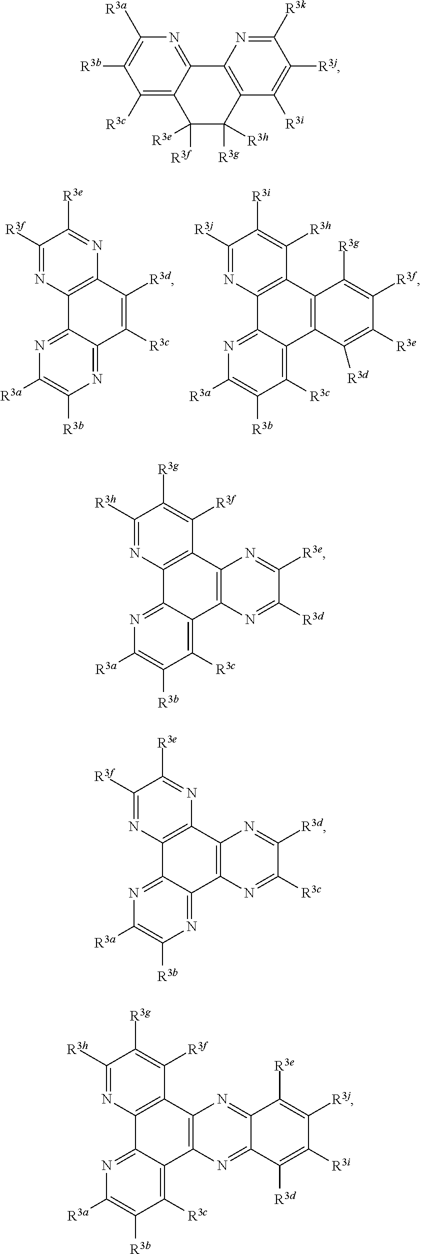 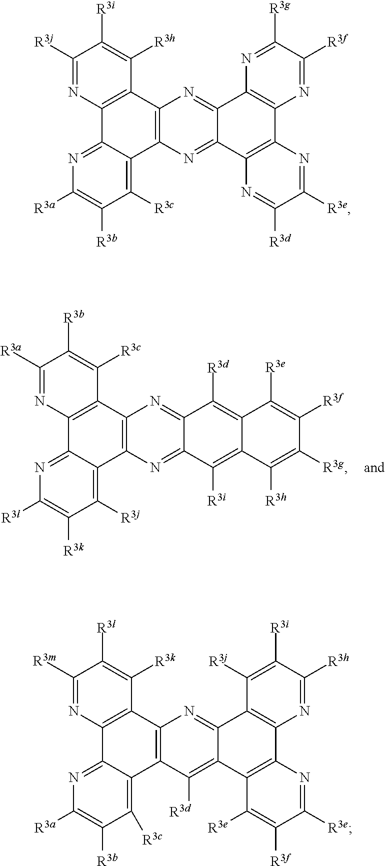
R¹ is selected from the group consisting of 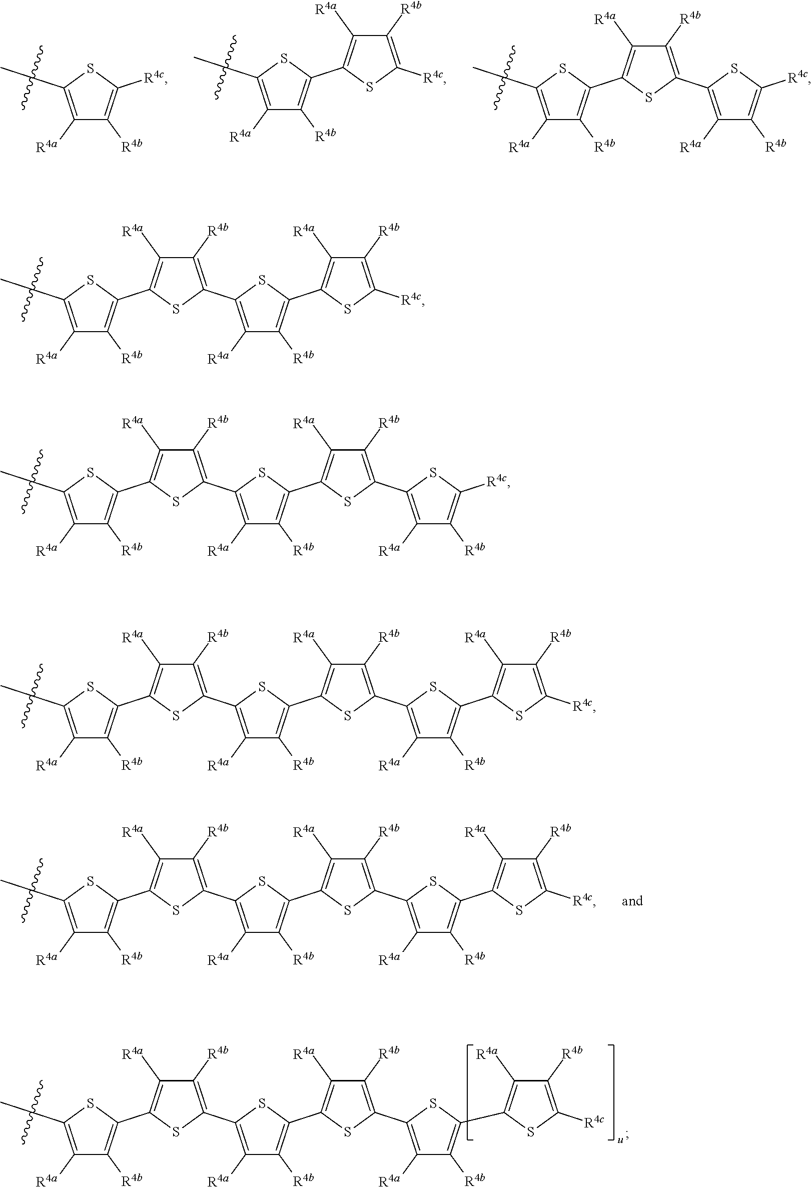
u is an integer;
R^{2a}, R^{2b}, R^{2c}, R^{2d}, R^{2e}, and R^{2f} at each occurrence are each independently selected from the group consisting of hydrogen, C₁₋₆ optionally substituted alkyl, C₁₋₆ optionally substituted branched alkyl, C₃₋₇ optionally substituted cycloalkyl, C₁₋₆ optionally substituted haloalkyl, C₁₋₆ optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h} R³ⁱ, R^{3j}, R^{3k}, R^{3l}, and R^{3m} at each occurrence are each independently selected from the group consisting of hydrogen, C₁₋₆ optionally substituted alkyl, C₁₋₆ optionally substituted branched alkyl, C₁₋₆ optionally substituted haloalkyl, C₁₋₆ optionally substituted alkoxy, and CO₂R⁸;
R^{4a}, R^{4b}, and R^{4c} at each occurrence are each independently selected from the group consisting of hydrogen, C₁₋₆ optionally substituted alkyl, C₁₋₆ optionally substituted branched alkyl, C₁₋₆ optionally substituted cycloalkyl, C₁₋₆ optionally substituted haloalkyl, C₁₋₆ optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence is independently selected from the group consisting of hydrogen and optionally substituted alkyl;
R⁶ at each occurrence is independently selected from the group consisting of hydrogen and optionally substituted alkyl;
R⁷ at each occurrence is independently selected from the group consisting of hydrogen and optionally substituted alkyl; and
R⁸ at each occurrence is independently selected from the group consisting of hydrogen and optionally substituted alkyl.

7. The method of claim 6, wherein the photodynamic compound is administered in combination with transferrin.

8. The method of claim 7, wherein the immunotherapeutic agent is at least one member selected from the group consisting of: idecabtagene vicleucel, lisocabtagene maraleucel, tisagenlecleucel, brexucabtagene autoleucel, axicabtagene ciloleucel, ciltacabtagene autoleucel, atezolizumab, avelumab, dostarlizumab, durvalumab, ipilimumab, nivolumab, pembrolizumab, pucotenlimab, pidilizumab, cemiplimab, spartalizumab, camrelizumab, cetrelimab, toripalimab, sintilimab, sasanlimab, dostarlizumab, SHR-1210, PDR001, MGA012, PDR001, AB122, AMP-224, JTX-4014, BGB-108, BCD-100, BAT1306, LZM009, AK105, HLX10, bevacizumab, trastuzumab, onartuzumab, erdafitinib, enapotamab, daratumumab, FAZ053, KN035, CS1001, SHR-1316, CBT-502, A167, STI-A101, CK-301, BGB-A333, MSB-2311, HLX20, LY3300054, sipuleucel-T, Bacillus Calmette-Guérin vaccine, nadofaragene firadonevec and T-VEC.

9. The method of claim 7, wherein the photodynamic compound is represented by formula (I) or formula (II).

10. The method of claim 5, wherein the photodynamic compound is at least one member selected from the group consisting of:
Ru(2,2′-bipyridine)₂(2-(2′,2″:5″,2′″-terthiophene)-imidazo[4,5-f][1,10]phenanthroline);
Ru(2,2′-bipyridine)₂(2-(2′,2″:5″,2′″;5′″,2″″-quaterthiophene)-imidazo[4,5-f][1,10]phenanthroline);
Ru(4,4′-dimethyl-2,2′-bipyridine)₂(2-thiophenimidazo[4,5-f][1,10]phenanthroline);
Ru(4,4′-dimethyl-2,2′-bipyridine)₂(2-(2′,2″-bithiophene)-imidazo[4,5-f][1,10]phenanthroline);
Ru(4,4′-dimethyl-2,2′-bipyridine)₂(2-(2′,2″:5″,2′″-terthiophene)-imidazo[4,5-f][1,10]phenanthroline);
Ru(4,4′-dimethyl-2,2′-bipyridine)₂(2-(2′,2″:5″,2′″;5′″,2″″-quaterthiophene)-imidazo[4,5-f][1,10]phenanthroline);
Ru(4,4′-di-t-butyl-2,2′-bipyridine)₂(2-thiophenimidazo[4,5-f][1,10]phenanthroline);
Ru(4,4′-di-t-butyl-2,2′-bipyridine)₂(2-(2′,2″-bithiophene)-imidazo[4,5-f][1,10]phenanthroline);
Ru(4,4′-di-t-butyl-2,2′-bipyridine)₂(2-(2′,2″:5″,2″-terthiophene)-imidazo[4,5-f][1,10]phenanthroline);
Ru(4,4′-dimethoxy-2,2′-bipyridine)₂(2-(2′,2″:5″,2″-terthiophene)-imidazo[4,5-f][1,10]phenanthroline);
Ru(5,5′-dimethyl-2,2′-bipyridine)₂(2-thiophenimidazo[4,5-f][1,10]phenanthroline);
Ru(5,5′-dimethyl-2,2′-bipyridine)₂(2-(2′,2″-bithiophene)-imidazo[4,5-f][1,10]phenanthroline);
Ru(5,5′-dimethyl-2,2′-bipyridine)₂(2-(2′,2″:5″,2′″-terthiophene)-imidazo[4,5-f][1,10]phenanthroline);
Ru(6,6′-dimethyl-2,2′-bipyridine)₂(2-thiophenimidazo[4,5-f][1,10]phenanthroline);
Ru(6,6′-dimethyl-2,2′-bipyridine)₂(2-(2′,2″-bithiophene)-imidazo[4,5-f][1,10]phenanthroline);
Ru(6,6′-dimethyl-2,2′-bipyridine)₂(2-(2′,2″:5″,2″-terthiophene)-imidazo[4,5-f][1,10]phenanthroline);
Ru(4,4′-di(methylcarboxy)-2,2′-bipyridine)₂(2-(2′,2″:5″,2′″-terthiophene)-imidazo[4,5-f][1,10]phenanthroline);
Ru(2,2′-bipyrimidine)₂(2-(2′,2″:5″,2′″-terthiophene)-imidazo[4,5-f][1,10]phenanthroline);
Ru(2,2′-bipyrimidine)(4,4′-dimethyl-2,2′-bipyridine)(2-(2′,2″:5″,2″-terthiophene)-imidazo[4,5-f][1,10]phenanthroline);
Ru(1,10-phenanthroline)₂(2-thiophenimidazo[4,5-f][1,10]phenanthroline);
Os(2,2′-bipyridine)₂(2-thiophenimidazo[4,5-f][1,10]phenanthroline);
Os(2,2′-bipyridine)₂(2-(2′,2″-bithiophene)-imidazo[4,5-f][1,10]phenanthroline);
Os(2,2′-bipyridine)₂(2-(2′,2″:5″,2′″-terthiophene)-imidazo[4,5-f][1,10]phenanthroline);
Os(1,10-phenanthroline)₂(2-thiophenimidazo[4,5-f][1,10]phenanthroline);
Os(1,10-phenanthroline)₂(2-(2′,2″-bithiophene)-imidazo[4,5-f][1,10]phenanthroline);
Os(1,10-phenanthroline)₂(2-(2′,2″:5″,2′″-terthiophene)-imidazo[4,5-f][1,10]phenanthroline);
Os(4,4′-dimethyl-2,2′-bipyridine)₂(2-thiophenimidazo[4,5-f][1,10]phenanthroline);
Os(4,4′-dimethyl-2,2′-bipyridine)₂(2-(2′,2″-bithiophene)-imidazo[4,5-f][1,10]phenanthroline);
Os(4,4′-dimethyl-2,2′-bipyridine)₂(2-(2′,2″:5″,2″″-terthiophene)-imidazo[4,5-f][1,10]phenanthroline); and
pharmaceutically acceptable salts thereof.

11. The method of claim 10, wherein the photodynamic compound is administered in combination with transferrin.

12. The method of claim 11, wherein the photodynamic compound is Ru(4,4′-dimethyl-2,2′-bipyridine)₂(2-(2′,2″:5″,2′-terthiophene)-imidazo[4,5-f][1,10]phenanthroline) or a pharmaceutically acceptable salt thereof.

13. The method of claim 1, wherein the radiation is infrared light, visible light, X-rays or other ionizing radiation.

14. The method of claim 1, wherein the immunotherapeutic agent, the photodynamic compound and the radiation are administered in amounts synergistically effective to treat the condition.

15. The method of claim 1, wherein the immunotherapeutic agent and the photodynamic compound are independently administered to the patient locally, intravesically, intratumorally or intravenously in an amount that ranges from a Maximum Recommended Starting Dose based on a Human Equivalent Dose to a Therapeutic Dose or a Biologically Effective Dose.

16. The method of claim 1, wherein step (a) is conducted before step (b) and step (b) is conducted before step (c).

17. The method of claim 2, wherein the cancer is characterized by expression of PD-1, PD-L1 or PD-L2.

18. The method of claim 2, wherein the cancer is selected from the group consisting of: bone cancer, pancreatic cancer, skin cancer, brain cancer, lung cancer, colorectal cancer, head cancer, neck cancer, cutaneous malignant melanoma, intraocular malignant melanoma, uterine cancer, ovarian cancer, rectal cancer, anal cancer, stomach cancer, testicular cancer, uterine cancer, fallopian tube carcinoma, carcinoma of the endometrium, endometrial cancer, carcinoma of the cervix, carcinoma of the vagina, carcinoma of the vulva, Hodgkin's Disease, non-Hodgkin's lymphoma, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, leukemia, solid tumors of childhood, lymphocytic lymphoma, cancer of the bladder, cancer of the kidney or urethra, carcinoma of the renal pelvis, neoplasm of the Central Nervous System (“CNS”), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancer, and combinations thereof.
